(19) United States
(12) Patent Application Publication
(10) Pub. No.: US 2025/0339208 A1
(43) Pub. Date: Nov. 6, 2025

(54) INTRALUMINAL ULTRASOUND IMAGING WITH AUTOMATIC DETECTION OF TARGET AND REFERENCE REGIONS

(71) Applicant: KONINKLIJKE PHILIPS N.V., EINDHOVEN (NL)

(72) Inventors: Nikhil Sreedhar RAJGURU, SAN DIEGO, CA (US); Anuja NAIR, HIGHLAND HEIGHTS, OH (US)

(21) Appl. No.: 18/868,848

(22) PCT Filed: Jun. 20, 2023
(86) PCT No.: PCT/EP2023/066518

§ 371 Date: Nov. 25, 2024

Related U.S. Application Data
(60) Provisional application No. 63/355,444, filed on Jun. 24, 2022.

(51) Int. Cl. A61B 34/20; A61B 8/00; A61B 8/08; A61B 8/12; G06T 7/00; G06T 7/62; G06T 7/70

(52) U.S. Cl. CPC A61B 34/20; A61B 8/0841; A61B 8/12; A61B 8/463; G06T 7/0012; G06T 7/62; G06T 7/70; A61B 2034/2063; G06T 2207/10132; G06T 2207/20081; G06T 2207/30052; G06T 2207/30101

(57) ABSTRACT

An intraluminal ultrasound imaging system is presented which includes a processor circuit configured for communication with an intraluminal ultrasound imaging catheter. The processor circuit is configured to receive a plurality of intraluminal ultrasound images obtained by the intraluminal ultrasound imaging catheter during movement within a body lumen of a patient. The body lumen includes a plurality of segments, as well as a compression within at least one segment. The processor circuit is also configured to automatically, based on the images, determine a target location within the compression and a reference location comprising a healthy portion of the lumen proximate to the compression; and output, to a display in communication with the processor circuit, a screen display that includes the target location, the reference location, and at least one quantity associated with the target location and reference location.

### TECHNICAL FIELD

The subject matter described herein relates to a system for medical imaging. In particular, the disclosed system provides a system for identifying treatment target and reference locations in peripheral intravascular ultrasound or IVUS images during a pullback procedure. This system has particular but not exclusive utility for diagnosis and treatment of vascular diseases.

### BACKGROUND

IVUS can be used to evaluate disease in peripheral vascular procedures and deep venous system procedures. Treatments may include stenting, IVC-filter retrieval, thrombectomy, and other procedures. Different diseases or medical procedures produce physical features with different size, structure, density, water content, and accessibility for imaging sensors. For example, a deep-vein thrombosis (DVT) produces a clot of blood cells, whereas post-thrombotic syndrome (PTS) produces webbing or other residual structural effects in a vessel that have similar composition to the vessel wall itself, and may thus be difficult to distinguish from the vessel wall. A stent is a dense (e.g., metallic) object that may be placed in a vessel or lumen to hold the vessel or lumen open to a particular diameter. A compression occurs when anatomical structures outside the vessel or lumen impinge on the vessel or lumen, constricting it.

In some cases, intraluminal medical imaging is carried out with an IVUS device including one or more ultrasound transducers. The IVUS device may be passed into the vessel and guided to the area to be imaged. The transducers emit ultrasonic energy and receive ultrasound echoes reflected from the vessel. The ultrasound echoes are processed to create an image of the vessel of interest. The image of the vessel of interest may include one or more lesions or blockages in the vessel. A stent may be placed within the vessel to treat these blockages and intraluminal imaging may be carried out to view the placement of the stent within the vessel. Other types of treatment include thrombectomy, ablation, angioplasty, pharmaceuticals, etc.

Interpretation of IVUS images of deep venous disease by a clinician is critical in identifying regions of compression or other disease, as well as corresponding normal or reference regions that are not diseased, and potential landing zones for stents or other treatments. However, considerable variation exists in the methods and thought processes used by clinicians for identifying these target and reference locations. This lack of standardization may result in varying outcomes, and may also make it difficult for novice clinicians to know which methods or thought processes to employ. The influence of external factors (e.g., reference anatomy from literature, a patient's age and disease history, etc.) and internal factors (e.g., the status of blood vessels surrounding the diseased vessel) may add additional complication to a clinician's thought process. For example, some studies show that approximately 1 in 3 physicians disagree with “normal” or reference anatomy in particular publications. Thus, IVUS interpretation can be very complex, and intermediate and beginner users may therefore not be confident in analyzing the pullbacks by themselves. Thus, in some settings there may be an overreliance on trained staff.

The information included in this Background section of the specification, including any references cited herein and any description or discussion thereof, is included for technical reference purposes only and is not to be regarded as subject matter by which the scope of the disclosure is to be bound.

### SUMMARY

Disclosed herein is an automatic target and reference detection system for intravascular ultrasound (IVUS) procedures treating peripheral veins or other vasculature. The present disclosure provides a uniform strategy for interpreting IVUS imagery of a deep venous pullback, while retaining flexibility for expert users to override the system's default behaviors. The systems, devices, and methods disclosed herein include algorithms that present target and reference locations to the user, including target frame(s) and reference frame(s) per segment of an iliofemoral pullback, as well as potential stent landing frames in pre therapy pullback, and/or areas of clinical interest in post-treatment pullbacks. The automatic target and reference detection system may also provide options for users to change various settings to support different thought processes. Thus, while the system supports standardization of deep venous IVUS procedures, it retains flexibility for experienced clinicians to use their own judgment.

Aspects of the present disclosure are particularly suitable for peripheral venous vasculature/system. In some instances, the peripheral venous vasculature can include or can be deep venous vasculature/system and/or peripheral deep venous vasculature/system. The peripheral venous vasculature can include a continuous length of veins, with different named vein segments (established be medical authorities) that are in fluid communication with one another to transport blood from the leg to the heart. For example, the peripheral venous vasculature can include veins in the abdomen and/or legs of a patient. The segments of the peripheral vasculature can include the inferior vena cava (IVC), iliac vein (e.g., common iliac vein, internal iliac vein, external iliac vein), femoral vein (e.g., common femoral vein, femoral vein), profunda femoris vein, popliteal vein, tibial vein, saphenous vein (e.g., great saphenous vein, small saphenous vein), and/or other veins (such as those illustrated in FIG. 2). With respect to peripheral venous vasculature, intravascular ultrasound (IVUS) is used to evaluate ilio-femoral disease, compression, etc., both pre- and post-procedure (e.g., stenting, IVC-filter retrieval, thrombectomy, and/or other procedures). Peripheral veins present different types of disease than coronary arteries. Accordingly, aspects of the present disclosure are particular well-suited in pre-treatment and post-treatment evaluation of diseases restricting blood flow in peripheral veins.

A system of one or more computers can be configured to perform particular operations or actions by virtue of having software, firmware, hardware, or a combination of them installed on the system that in operation causes or cause the system to perform the actions. For example, any one or plurality of modules and/or steps described herein can implemented by hardware and/or software in a processor circuit and/or processor. One or more computer programs can be configured to perform particular operations or actions by virtue of including instructions that, when executed by data processing apparatus, cause the apparatus to perform the actions. One general aspect includes an intraluminal ultrasound imaging system including a processor circuit configured for communication with an intraluminal ultrasound imaging catheter. The processor circuit is configured to: receive a first plurality of intraluminal ultrasound images obtained by the intraluminal ultrasound imaging catheter during movement of the intraluminal ultrasound imaging catheter within a body lumen of a patient, the body lumen including a plurality of segments and a compression within at least one segment; for at least one segment, based on the first plurality of intraluminal ultrasound images, automatically determine a target location within the compression and a reference location including a healthy portion of the lumen proximate to the compression; and output, to a display in communication with the processor circuit, a screen display including the target location, the reference location, and at least one quantity associated with the target location and reference location. Other embodiments of this aspect include corresponding computer systems, apparatus, and computer programs recorded on one or more computer storage devices, each configured to perform the actions of the methods.

Implementations may include one or more of the following features. In some embodiments, the processor circuit is further configured to: receive a second plurality of intraluminal ultrasound images obtained by the intraluminal ultrasound imaging catheter during movement of the intraluminal ultrasound imaging catheter within the body lumen, the body lumen including a stent aligned longitudinally with the target location; based on the second plurality of intraluminal ultrasound images, automatically determine a proximal end of the stent, a distal end of the stent, and a location within the stent where the stent is most constricted; and output, to the screen display, the proximal end of the stent, the distal end of the stent, the location where the stent is most constricted, and at least one quantity associated with the constriction. In some embodiments, automatically determining at least one of the target location, the reference location, the proximal end of the stent, the distal end of the stent, or the location within the stent where the stent is most constricted involves a machine learning algorithm. In some embodiments, the body lumen includes peripheral vasculature and where the plurality of segments includes at least one of a common iliac vein (CIV), an external iliac vein (EIV), a common femoral vein (CFV), or a femoral vein for popliteal access. In some embodiments, the at least one segment includes multiple segments. In some embodiments, the screen display simultaneously shows the target frame and reference frame for each of the multiple segments. In some embodiments, the target location is determined at least in part by variation of a first lumen metric along the segment, and the reference location is determined at least in part by variation of a second lumen metric along the segment. In some embodiments, the first lumen metric and the second lumen metric are the same. In some embodiments, the first lumen metric or the second lumen metric includes an area, minimum diameter, max diameter, effective diameter, average diameter, aspect ratio, or flow resistance of the lumen. In some embodiments, the target location or the reference location is determined based at least in part on the intraluminal ultrasound imaging catheter. In some embodiments, the target location or the reference location is determined based at least in part on a length of the movement of the intraluminal ultrasound imaging catheter within the body lumen. In some embodiments, the target location or the reference location is determined based at least in part on the segment in which the compression is located. In some embodiments, the target location or the reference location is determined based at least in part on a border of a second body lumen adjacent to the body lumen. In some embodiments, the screen display includes at least one of a roadmap image or an image longitudinal display (ILD). Implementations of the described techniques may include hardware, a method or process, or computer software on a computer-accessible medium.

One general aspect includes an intraluminal ultrasound imaging method with a processor circuit in communication with an intraluminal ultrasound imaging catheter: receiving a first plurality of intraluminal ultrasound images obtained by the intraluminal ultrasound imaging catheter during movement of the intraluminal ultrasound imaging catheter within a body lumen of a patient, the body lumen including a plurality of segments and a compression within at least one segment; for at least one segment, based on the first plurality of intraluminal ultrasound images, automatically determining a target location within the compression and a reference location including a healthy portion of the lumen proximate to the compression; and outputting, to a display in communication with the processor circuit, a screen display including the target location, the reference location, and at least one quantity associated with the target location and reference location. Other embodiments of this aspect include corresponding computer systems, apparatus, and computer programs recorded on one or more computer storage devices, each configured to perform the actions of the methods.

Implementations may include one or more of the following features. In some embodiments, The method further includes, with the processor circuit: receiving a second plurality of intraluminal ultrasound images obtained by the intraluminal ultrasound imaging catheter during movement of the intraluminal ultrasound imaging catheter within the body lumen, the body lumen including a stent aligned longitudinally with the target location; based on the second plurality of intraluminal ultrasound images, automatically determining a proximal end of the stent, a distal end of the stent, and a location within the stent where the stent is most constricted; and outputting, to the screen display, the proximal end of the stent, the distal end of the stent, the location where the stent is most constricted, and at least one quantity associated with the constriction. In some embodiments, the body lumen includes peripheral vasculature and where the plurality of segments includes at least one of a common iliac vein (civ), an external iliac vein (eiv), or a common femoral vein (cfv). In some embodiments, the at least one segment includes multiple segments, and the screen display simultaneously shows the target frame and reference frame for each of the multiple segments. In some embodiments, the target location is determined at least in part by variation of a first lumen metric along the segment, and the reference location is determined at least in part by variation of a second lumen metric along the segment, where the first lumen metric or the second lumen metric includes an area, minimum diameter, max diameter, effective diameter, average diameter, aspect ratio, or flow resistance of the lumen. In some embodiments, the first lumen metric and the second lumen metric are the same. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of the automatic target and reference detection system, as defined in the claims, is provided in the following written description of various embodiments of the disclosure and illustrated in the accompanying drawings.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 illustrates blood vessels (e.g., arteries and veins) in the human body.

FIG. 3 illustrates a blood vessel incorporating a compression.

FIG. 4 illustrates a blood vessel incorporating a compression and with a stent expanded inside it to restore flow.

FIG. 5 illustrates an example intraluminal imaging display screen in accordance with at least one embodiment of the present disclosure.

FIG. 6 is a schematic, diagrammatic representation of a clinician's thought process, in accordance with at least one embodiment of the present disclosure.

FIG. 7 is a diagrammatic representation, in flow diagram form, of an example pre-treatment reference frame detection method, in accordance with at least one embodiment of the present disclosure.

FIG. 8 is a diagrammatic representation, in flow diagram form, of an example post-treatment stent inspection method, in accordance with at least one embodiment of the present disclosure.

FIG. 9 is a diagrammatic representation, in block diagram form, of at least a portion of an example pre-treatment automatic target and reference detection system or method, in accordance with at least one embodiment of the present disclosure.

FIG. 10 is a diagrammatic representation, in block diagram form, of at least a portion of an example pre-treatment automatic target and reference detection system or method, in accordance with at least one embodiment of the present disclosure.

FIG. 11 is a diagrammatic representation, in block diagram form, of at least a portion of an example post-treatment automatic stent evaluation system or method, in accordance with at least one embodiment of the present disclosure.

FIG. 12 is a diagrammatic representation, in block diagram form, of at least a portion of an example post-treatment automatic stent evaluation system or method, in accordance with at least one embodiment of the present disclosure.

FIG. 13 is a diagrammatic representation, in block diagram form, of at least a portion of an example pre- and post-treatment automatic target identification, reference identification, and stent evaluation system or method, in accordance with at least one embodiment of the present disclosure.

FIG. 14 is a schematic diagram of a processor circuit, in accordance with at least one embodiment of the present disclosure.

FIG. 15 is a diagrammatic representation, in flow diagram form, of an example pre-treatment target zone and reference frame detection method, in accordance with at least one embodiment of the present disclosure.

FIG. 16 is a schematic, diagrammatic representation of the analyzed IVUS image data, in accordance with at least one embodiment of the present disclosure.

FIG. 17 illustrates an example intraluminal imaging display screen, in accordance with at least one embodiment of the present disclosure.

FIG. 18 is a diagrammatic representation, in flow diagram form, of an example pre-treatment target zone and reference frame detection method, in accordance with at least one embodiment of the present disclosure.

### DETAILED DESCRIPTION

The present disclosure relates generally to medical imaging, including imaging associated with a body lumen of a patient using an intraluminal imaging device. For example, the present disclosure describes systems, devices, and methods for detecting treatment target locations and healthy reference locations in peripheral veins or other vasculature.

Considerable variation exists in the methods and thought processes used by clinicians for identifying these target and reference locations, as well as landing zones for stents or other treatments. Training of clinicians can therefore be difficult and inconsistent. To address this need, the present disclosure provides a common strategy for interpreting IVUS imagery of a deep venous pullback, while retaining flexibility for expert users to override the system's default behaviors.

The application logic disclosed herein includes algorithms that present target and reference locations (e.g., particular IVUS image frames) to the user. The overall algorithm describes how to find a target frame (or frames), a reference frame (or frames) per segment of the iliofemoral pullback. In some embodiments, the analysis performed by the algorithm also includes finding potential stent landing frames in pre therapy pullback, and/or areas of clinical interest in post-treatment pullbacks. The automatic target and reference detection system may also provide options for users to change various settings to support different thought processes. Thus, while the system supports standardization of deep venous IVUS procedures, it retains flexibility for experienced clinicians to use their own judgment. Thus, a goal of the system is to present users with relevant information about the high-level pullback analysis—not to tell the user what to treat. The algorithm may employ reference values, ratios, averages, means, or formulae from published studies, in order to facilitate acceptance by clinicians while standardizing outcomes.

The devices, systems, and methods described herein can include one or more features described in U.S. Provisional App. No. 62/946,097 (Attorney Docket No. 2018PF01110-44755.2066PV01), filed Dec. 10, 2019, U.S. Provisional App. No. 63/250,498 (Attorney Docket No. 2021PF00350-44755.2223PV01), filed Sep. 30, 2021, U.S. Provisional App. No. 62/750,983 (Attorney Docket No. 2018PF01112-44755.1996PV01), filed Oct. 26, 2018, U.S. Provisional App. No. 62/750,983 (Attorney Docket No. 2018PF01112-44755.2000PV01), filed 26 Oct. 2018, U.S. Provisional App. No. 62/751,268 (Attorney Docket No. 2018PF01160-44755.1997PV01), filed 26 Oct. 2018, U.S. Provisional App. No. 62/751,289 (Attorney Docket No. 2018PF01159-44755.1998PV01), filed 26 Oct. 2018, U.S. Provisional App. No. 62/750,996 (Attorney Docket No. 2018PF01145-44755.1999PV01), filed 26 Oct. 2018, U.S. Provisional App. No. 62/751,167 (Attorney Docket No. 2018PF01115-44755.2000PV01), filed 26 Oct. 2018, and U.S. Provisional App. No. 62/751,185 (Attorney Docket No. 2018PF01116-44755.2001PV01), filed 26 Oct. 2018, each of which is hereby incorporated by reference in its entirety as though fully set forth herein.

The devices, systems, and methods described herein can also include one or more features described in U.S. Provisional App. No. 62/642,847 (Attorney Docket No. 2017PF02103), filed Mar. 14, 2018 (and a Non-Provisional Application filed therefrom on Mar. 12, 2019 as U.S. Ser. No. 16/351,175), U.S. Provisional App. No. 62/712,009 (Attorney Docket No. 2017PF02296), filed Jul. 30, 2018, U.S. Provisional App. No. 62/711,927 (Attorney Docket No. 2017PF02101), filed Jul. 30, 2018, and U.S. Provisional App. No. 62/643,366 (Attorney Docket No. 2017PF02365), filed Mar. 15, 2018 (and a Non-Provisional Application filed therefrom on Mar. 15, 2019 as U.S. Ser. No. 16/354,970), each of which is hereby incorporated by reference in its entirety as though fully set forth herein.

The present disclosure substantially aids a clinician in making sense of large volumes of intraluminal imaging data, along with reporting and treatment planning, plus reduced case time and improved ease of use. The present disclosure accomplishes this by providing a quick, seamless process for identification and marking of locations of interest within a vessel or lumen along an examined length, in real time during the imaging procedure (e.g., an IVUS pullback procedure). Implemented on a medical imaging console (e.g., an IVUS imaging console) in communication with a medical imaging sensor (e.g., an intraluminal ultrasound sensor), the automatic target and reference detection system disclosed herein provides both time savings and an improvement in the accuracy of bookmarking of captured images. This improved imaging workflow transforms a time-consuming process of imaging, image selection, review, and clinical judgement into a streamlined, repeatable process involving both fewer steps and simpler steps on the part of the clinician. This occurs for example without the normally routine need for a clinician to perform mathematical calculations or apply visual judgment in identifying target, reference, and landing locations. This unconventional approach improves the functioning of the medical imaging console and sensor, by automating bookmarking steps that are normally performed manually by the clinician or other users.

The automatic target and reference detection system may be implemented as a set of logical branches and mathematical operations, whose outputs are viewable on a display, and operated by a control process executing on a processor that accepts user inputs (e.g., from a user interface such as a keyboard, mouse, or touchscreen interface), and that is in communication with one or more medical imaging sensors (e.g., intraluminal ultrasound sensors). In that regard, the control process performs certain specific operations in response to different inputs or selections made by a user at the start of an imaging procedure, and may also respond to inputs made by the user during the procedure. Certain structures, functions, and operations of the processor, display, sensors, and user input systems are known in the art, while others are recited herein to enable novel features or aspects of the present disclosure with particularity.

Various types of intraluminal imaging systems are used in diagnosing and treating diseases. For example, intravascular ultrasound (IVUS) imaging is used as a diagnostic tool for visualizing vessels within a body of a patient. This may aid in assessing diseased or compressed vessels, such as arteries or veins, within the human body to determine the need for treatment, to optimize treatment, and/or to assess a treatment's effectiveness (e.g., through imaging of the vessel before and after treatment).

In some cases, intraluminal imaging is carried out with an IVUS device including one or more ultrasound transducers. The IVUS device may be passed into the vessel and guided to the area to be imaged. The transducers emit ultrasonic energy and receive ultrasound echoes reflected from the vessel. The ultrasound echoes are processed to create an image of the vessel of interest. The image of the vessel of interest may include one or more lesions or blockages in the vessel. A stent may be placed within the vessel to treat these blockages and intraluminal imaging may be carried out to view the placement of the stent within the vessel. Other types of treatment include thrombectomy, ablation, angioplasty, pharmaceuticals, etc.

These descriptions are provided for exemplary purposes only, and should not be considered to limit the scope of the automatic target and reference detection system. Certain features may be added, removed, or modified without departing from the spirit of the claimed subject matter.

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

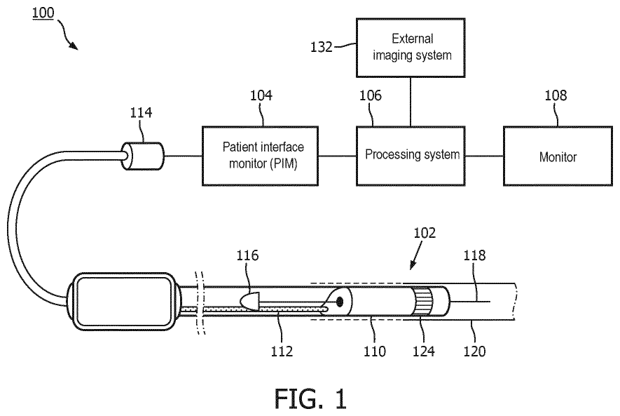

FIG. 1 is a diagrammatic schematic view of an intraluminal imaging system incorporating the automatic target and reference detection system, according to aspects of the present disclosure. The intraluminal imaging system 100 can be an intravascular ultrasound (IVUS) imaging system in some embodiments. The intraluminal imaging system 100 may include an intraluminal device 102, a patient interface module (PIM) 104, a console or processing system 106, a monitor 108, and an external imaging system 132 which may include angiography, ultrasound, X-ray, computed tomography (CT), magnetic resonance imaging (MRI), or other imaging technologies, equipment, and methods. The intraluminal device 102 is sized and shaped, and/or otherwise structurally arranged to be positioned within a body lumen of a patient. For example, the intraluminal device 102 can be a catheter, guide wire, guide catheter, pressure wire, and/or flow wire in various embodiments. In some circumstances, the system 100 may include additional elements and/or may be implemented without one or more of the elements illustrated in FIG. 1. For example, the system 100 may omit the external imaging system 132.

The intraluminal imaging system 100 (or intravascular imaging system) can be any type of imaging system suitable for use in the lumens or vasculature of a patient. In some embodiments, the intraluminal imaging system 100 is an intraluminal ultrasound (IVUS) imaging system. In other embodiments, the intraluminal imaging system 100 may include systems configured for forward looking intraluminal ultrasound (FL-IVUS) imaging, intraluminal photoacoustic (IVPA) imaging, intracardiac echocardiography (ICE), transesophageal echocardiography (TEE), and/or other suitable imaging modalities.

It is understood that the system 100 and/or device 102 can be configured to obtain any suitable intraluminal imaging data. In some embodiments, the device 102 may include an imaging component of any suitable imaging modality, such as optical imaging, optical coherence tomography (OCT), etc. In some embodiments, the device 102 may include any suitable non-imaging component, including a pressure sensor, a flow sensor, a temperature sensor, an optical fiber, a reflector, a mirror, a prism, an ablation element, a radio frequency (RF) electrode, a conductor, or combinations thereof. Generally, the device 102 can include an imaging element to obtain intraluminal imaging data associated with the lumen 120. The device 102 may be sized and shaped (and/or configured) for insertion into a vessel or lumen 120 of the patient.

The system 100 may be deployed in a catheterization laboratory having a control room. The processing system 106 may be located in the control room. Optionally, the processing system 106 may be located elsewhere, such as in the catheterization laboratory itself. The catheterization laboratory may include a sterile field while its associated control room may or may not be sterile depending on the procedure to be performed and/or on the health care facility. The catheterization laboratory and control room may be used to perform any number of medical imaging procedures such as angiography, fluoroscopy, CT, IVUS, virtual histology (VH), forward looking IVUS (FL-IVUS), intraluminal photoacoustic (IVPA) imaging, a fractional flow reserve (FFR) determination, a coronary flow reserve (CFR) determination, optical coherence tomography (OCT), computed tomography, intracardiac echocardiography (ICE), forward-looking ICE (FLICE), intraluminal palpography, transesophageal ultrasound, fluoroscopy, and other medical imaging modalities, or combinations thereof. In some embodiments, device 102 may be controlled from a remote location such as the control room, such than an operator is not required to be in close proximity to the patient.

The intraluminal device 102, PIM 104, monitor 108, and external imaging system 132 may be communicatively coupled directly or indirectly to the processing system 106. These elements may be communicatively coupled to the medical processing system 106 via a wired connection such as a standard copper link or a fiber optic link and/or via wireless connections using IEEE 802.11 Wi-Fi standards, Ultra Wide-Band (UWB) standards, wireless FireWire, wireless USB, or another high-speed wireless networking standard. The processing system 106 may be communicatively coupled to one or more data networks, e.g., a TCP/IP-based local area network (LAN). In other embodiments, different protocols may be utilized such as Synchronous Optical Networking (SONET). In some cases, the processing system 106 may be communicatively coupled to a wide area network (WAN). The processing system 106 may utilize network connectivity to access various resources. For example, the processing system 106 may communicate with a Digital Imaging and Communications in Medicine (DICOM) system, a Picture Archiving and Communication System (PACS), and/or a Hospital Information System (HIS) via a network connection.

At a high level, an ultrasound imaging intraluminal device 102 emits ultrasonic energy from a transducer array 124 included in scanner assembly 110 mounted near a distal end of the intraluminal device 102. The ultrasonic energy is reflected by tissue structures in the medium (such as a lumen 120) surrounding the scanner assembly 110, and the ultrasound echo signals are received by the transducer array 124. The scanner assembly 110 generates electrical signal(s) representative of the ultrasound echoes. The scanner assembly 110 can include one or more single ultrasound transducers and/or a transducer array 124 in any suitable configuration, such as a planar array, a curved array, a circumferential array, an annular array, etc. For example, the scanner assembly 110 can be a one-dimensional array or a two-dimensional array in some instances. In some instances, the scanner assembly 110 can be a rotational ultrasound device. The active area of the scanner assembly 110 can include one or more transducer materials and/or one or more segments of ultrasound elements (e.g., one or more rows, one or more columns, and/or one or more orientations) that can be uniformly or independently controlled and activated. The active area of the scanner assembly 110 can be patterned or structured in various basic or complex geometries. The scanner assembly 110 can be disposed in a side-looking orientation (e.g., ultrasonic energy emitted perpendicular and/or orthogonal to the longitudinal axis of the intraluminal device 102) and/or a forward-looking looking orientation (e.g., ultrasonic energy emitted parallel to and/or along the longitudinal axis). In some instances, the scanner assembly 110 is structurally arranged to emit and/or receive ultrasonic energy at an oblique angle relative to the longitudinal axis, in a proximal or distal direction. In some embodiments, ultrasonic energy emission can be electronically steered by selective triggering of one or more transducer elements of the scanner assembly 110.

The ultrasound transducer(s) of the scanner assembly 110 can be a piezoelectric micromachined ultrasound transducer (PMUT), capacitive micromachined ultrasonic transducer (CMUT), single crystal, lead zirconate titanate (PZT), PZT composite, other suitable transducer type, and/or combinations thereof. In an embodiment the ultrasound transducer array 124 can include any suitable number of individual transducer elements or acoustic elements between 1 acoustic element and 1000 acoustic elements, including values such as 2 acoustic elements, 4 acoustic elements, 36 acoustic elements, 64 acoustic elements, 128 acoustic elements, 500 acoustic elements, 812 acoustic elements, and/or other values both larger and smaller.

The PIM 104 transfers the received echo signals to the processing system 106 where the ultrasound image (including the flow information) is reconstructed and displayed on the monitor 108. The console or processing system 106 can include a processor and a memory. The processing system 106 may be operable to facilitate the features of the intraluminal imaging system 100 described herein. For example, the processor can execute computer readable instructions stored on the non-transitory tangible computer readable medium.

The PIM 104 facilitates communication of signals between the processing system 106 and the scanner assembly 110 included in the intraluminal device 102. This communication may include providing commands to integrated circuit controller chip(s) within the intraluminal device 102, selecting particular element(s) on the transducer array 124 to be used for transmit and receive, providing the transmit trigger signals to the integrated circuit controller chip(s) to activate the transmitter circuitry to generate an electrical pulse to excite the selected transducer array element(s), and/or accepting amplified echo signals received from the selected transducer array element(s) via amplifiers included on the integrated circuit controller chip(s). In some embodiments, the PIM 104 performs preliminary processing of the echo data prior to relaying the data to the processing system 106. In examples of such embodiments, the PIM 104 performs amplification, filtering, and/or aggregating of the data. In an embodiment, the PIM 104 also supplies high- and low-voltage DC power to support operation of the intraluminal device 102 including circuitry within the scanner assembly 110.

The processing system 106 receives echo data from the scanner assembly 110 by way of the PIM 104 and processes the data to reconstruct an image of the tissue structures in the medium surrounding the scanner assembly 110. Generally, the device 102 can be utilized within any suitable anatomy and/or body lumen of the patient. The processing system 106 outputs image data such that an image of the vessel or lumen 120, such as a cross-sectional IVUS image of the lumen 120, is displayed on the monitor 108. Lumen 120 may represent fluid filled or fluid-surrounded structures, both natural and man-made. Lumen 120 may be within a body of a patient. Lumen 120 may be a blood vessel, such as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

The controller or processing system 106 may include a processing circuit having one or more processors in communication with memory and/or other suitable tangible computer readable storage media. The controller or processing system 106 may be configured to carry out one or more aspects of the present disclosure. In some embodiments, the processing system 106 and the monitor 108 are separate components. In other embodiments, the processing system 106 and the monitor 108 are integrated in a single component. For example, the system 100 can include a touch screen device, including a housing having a touch screen display and a processor. The system 100 can include any suitable input device, such as a touch sensitive pad or touch screen display, keyboard/mouse, joystick, button, etc., for a user to select options shown on the monitor 108. The processing system 106, the monitor 108, the input device, and/or combinations thereof can be referenced as a controller of the system 100. The controller can be in communication with the device 102, the PIM 104, the processing system 106, the monitor 108, the input device, and/or other components of the system 100.

In some embodiments, the intraluminal device 102 includes some features similar to traditional solid-state IVUS catheters, such as the EagleEye® catheter available from Volcano Corporation and those disclosed in U.S. Pat. No. 7,846,101 hereby incorporated by reference in its entirety. For example, the intraluminal device 102 may include the scanner assembly 110 near a distal end of the intraluminal device 102 and a transmission line bundle 112 extending along the longitudinal body of the intraluminal device 102. The cable or transmission line bundle 112 can include a plurality of conductors, including one, two, three, four, five, six, seven, or more conductors.

The transmission line bundle 112 terminates in a PIM connector 114 at a proximal end of the intraluminal device 102. The PIM connector 114 electrically couples the transmission line bundle 112 to the PIM 104 and physically couples the intraluminal device 102 to the PIM 104. In an embodiment, the intraluminal device 102 further includes a guidewire exit port 116. Accordingly, in some instances the intraluminal device 102 is a rapid-exchange catheter. The guidewire exit port 116 allows a guidewire 118 to be inserted towards the distal end in order to direct the intraluminal device 102 through the lumen 120.

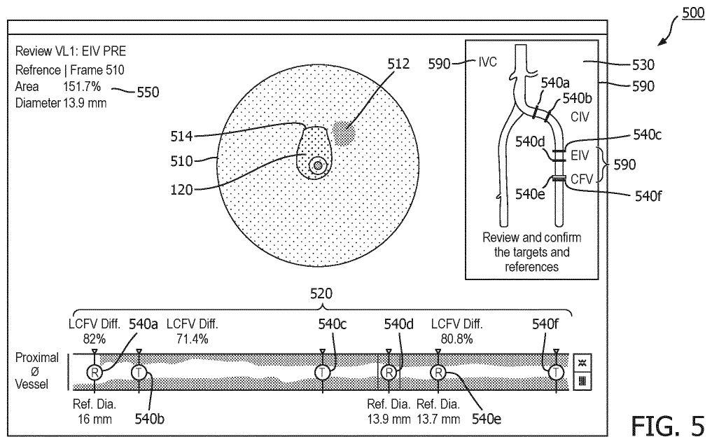

The monitor 108 may be a display device such as a computer monitor or other type of screen. The monitor 108 may be used to display selectable prompts, instructions, and visualizations of imaging data to a user. In some embodiments, the monitor 108 may be used to provide a procedure-specific workflow to a user to complete an intraluminal imaging procedure. This workflow may include performing a pre-stent plan to determine the state of a lumen and potential for a stent, as well as a post-stent inspection to determine the status of a stent that has been positioned in a lumen. The workflow may be presented to a user as any of the displays or visualizations shown in FIGS. 5-11.

The external imaging system 132 can be configured to obtain x-ray, radiographic, angiographic/venographic (e.g., with contrast), and/or fluoroscopic (e.g., without contrast) images of the body of a patient (including the vessel 120). External imaging system 132 may also be configured to obtain computed tomography images of the body of the patient (including the vessel 120). The external imaging system 132 may include an external ultrasound probe configured to obtain ultrasound images of the body of the patient (including the vessel 120) while positioned outside the body. In some embodiments, the system 100 includes other imaging modality systems (e.g., MRI) to obtain images of the body of the patient (including the vessel 120). The processing system 106 can utilize the images of the body of the patient in conjunction with the intraluminal images obtained by the intraluminal device 102.

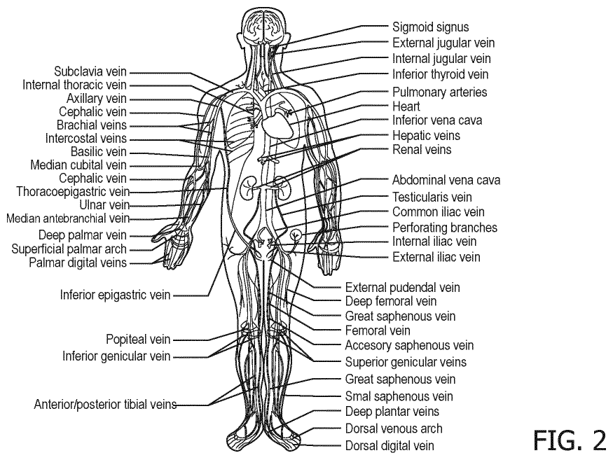

FIG. 2 illustrates blood vessels (e.g., arteries and veins) in the human body. For example, veins of the human body are labeled. Aspects of the present disclosure can be related to peripheral vasculature, e.g., veins in the torso or legs.

Occlusions can occur in arteries or veins. An occlusion can be generally representative of any blockage or other structural arrangement that results in a restriction to the flow of fluid through the lumen (e.g., an artery or a vein), for example, in a manner that is deleterious to the health of the patient. For example, the occlusion narrows the lumen such that the cross-sectional area of the lumen and/or the available space for fluid to flow through the lumen is decreased. Where the anatomy is a blood vessel, the occlusion may be a result of narrowing due to compression (e.g. from external vessels), plaque buildup, including without limitation plaque components such as fibrous, fibro-lipidic (fibro fatty), necrotic core, calcified (dense calcium), blood, and/or different stages of thrombus (e.g., acute, sub-acute, chronic, etc.). In some instances, the occlusion can be referenced as thrombus, a stenosis, and/or a lesion. Generally, the composition of the occlusion will depend on the type of anatomy being evaluated. Healthier portions of the anatomy may have a uniform or symmetrical profile (e.g., a cylindrical profile with a circular cross-sectional profile). The occlusion may not have a uniform or symmetrical profile. Accordingly, diseased or compressed portions of the anatomy, with the occlusion, will have a non-symmetric and/or otherwise irregular profile. The anatomy can have one occlusion or multiple occlusions.

Build-up of occlusion (e.g., thrombus, deep vein thrombosis or DVT, chronic total occlusion or CTO, etc.) is one way in which the cross-sectional area of the vein in the peripheral vasculature (e.g., torso, abdomen, groin, leg) may be reduced. Other anatomy that contacts the vein can also reduce its cross-sectional area, thereby restricting blood flow therethrough. For example, arteries or ligaments in the torso, abdomen, groin, or leg can press against a vein, which changes the shape of the vein and reduces its cross-sectional area. Such reductions in cross-sectional area resulting from contact with other anatomy can be referenced as compression, in that the walls of the vein are compressed as a result of the contact with the artery or ligament.

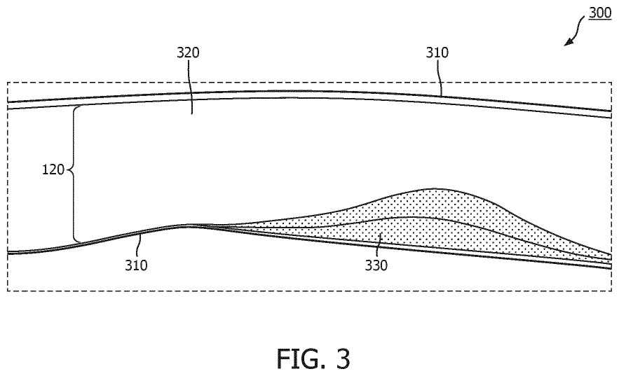

FIG. 3 illustrates a blood vessel 300 incorporating a compression 330. The compression 330 occurs outside the vessel walls 310 and may restrict the flow of blood 320. The compression may be caused by other anatomical structures outside the blood vessel 300, including but not limited to a tendon, ligament, or neighboring lumen.

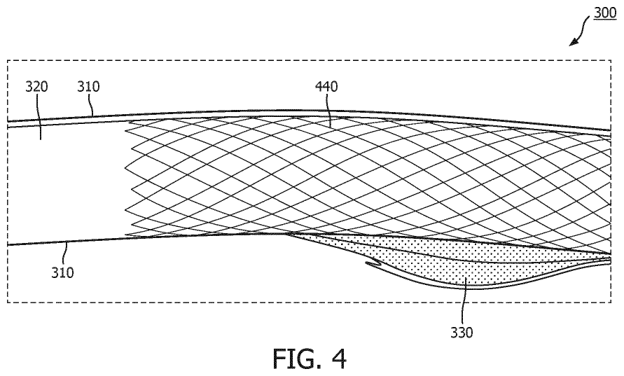

FIG. 4 illustrates a blood vessel 300 incorporating a compression 330 and with a stent 440 expanded inside it to restore flow. The stent 440 displaces and arrests the compression 330, pushing the vessel walls 310 outward, thus reducing the flow restriction for the blood 320. Other treatment options for alleviating an occlusion may include but are not limited to thrombectomy, ablation, angioplasty, and pharmaceuticals. However, in a large majority of cases it may be highly desirable to obtain accurate and timely intravascular images of the affected area, along with accurate and detailed knowledge of the location, orientation, length, and volume of the affected area prior to, during, or after treatment.

FIG. 5 illustrates an example intraluminal imaging display screen 500 in accordance with at least one embodiment of the present disclosure. In this example, the screen display 500 includes a current tomographic IVUS image 510 from a series of successive tomographic images, an Image Longitudinal Display (ILD) 520 containing stacked longitudinal cross-sections of the series of successive tomographic images, and a graphical roadmap 530. Also visible are bookmarks 540a, 540b, 540c, 540d, 540e, and 540f, that are associated with both the graphical roadmap 530 and the ILD 520. Bookmark 540d is also associated with the current IVUS image 510, as is a label 550 that contains information about the location and nature of the IVUS image 510. In this example, the IVUS image is identified as a reference image of the left external iliac vein. In addition, the bookmark information can be saved to reports that are automatically generated. If a change to the bookmark is made in any of these locations, the automatic target and reference detection system updates the bookmark in all of these locations, thus saving time and simplifying the process of identifying target and reference frames.

Bookmark 540a represents a reference location within the left common iliac vein (CIV). Bookmark 540b represents a target location within the left CIV. Bookmark 540c represents a target location within the left external iliac vein (EIV). Bookmark 540d represents a reference location within the left EIV. Bookmark 540e represents a reference location within the left common femoral vein (CFV). Bookmark 540f represents a target location within the left CFV. Other segments may be identified by the system, including but not limited to the inferior vena cava (IVC), a femoral vein (e.g., one used for popliteal access), an iliac vein, etc.

It is understood that in some cases, one or more segments of the vein (e.g., the CIV, EIV, CFV, etc.) may have multiple target and/or reference locations, as may occur for example if more than one compression or other occlusion is present in that segment. Similarly, in some cases, one or more segments of the vein may have no compressions or other occlusions, and may thus have no identified target or reference locations. In some circumstances, the reference location may also mark the landing zone for a stent, although this may not always be the case.

Other vessel segments or lumen segments may be identified in other areas of the body. In some embodiments, the identification of vessel segments is performed automatically by the automatic target and reference detection system (e.g., using image recognition, speed tracking, and position estimation). In other embodiments, bookmarks are predictively suggested to the clinician or other user. Predicting the next bookmark that the user will need advantageously avoids a requirement for the user to look through a list of bookmarks to find the correct one, or type in a manual bookmark. In other embodiments, the identification of vessel segments is performed by a clinician or other user with the assistance of the automatic target and reference detection system. Bookmarks or labels can be applied for example to a location where the segment begins or ends, or another segment begins or ends.

A bookmark may be automatically associated with an intraluminal image 510 that occurs at that location, and also with the corresponding locations on the ILD 520 and graphical roadmap 530. In addition, the automatic target and reference detection system may automatically populate a label 590 that is automatically associated with the bookmark and the associated intraluminal image and may include, for example, the bookmark information, Segment name, frame number, and image type (e.g., reference, pre-treatment target, post-treatment target, etc.) These steps may be performed automatically by the automatic target and reference detection system, without the need for user input of any kind, based on image recognition to track known bifurcations of a vessel or lumen as anatomic landmarks. Bookmarks may also be suggested or automatically placed based on automated image recognition of issues such as thrombus, webbing, and compression (venous) or stenosis (arterial).

Examples of border detection, image processing, image analysis, and/or pattern recognition include U.S. Pat. No. 6,200,268 entitled “VASCULAR PLAQUE CHARACTERIZATION” issued Mar. 13, 2001 with D. Geoffrey Vince, Barry D. Kuban and Anuja Nair as inventors, U.S. Pat. No. 6,381,350 entitled “INTRAVASCULAR ULTRASONIC ANALYSIS USING ACTIVE CONTOUR METHOD AND SYSTEM” issued Apr. 30, 2002 with Jon D. Klingensmith, D. Geoffrey Vince and Raj Shekhar as inventors, U.S. Pat. No. 7,074,188 entitled “SYSTEM AND METHOD OF CHARACTERIZING VASCULAR TISSUE” issued Jul. 11, 2006 with Anuja Nair, D. Geoffrey Vince, Jon D. Klingensmith and Barry D. Kuban as inventors, U.S. Pat. No. 7,175,597 entitled “NON-INVASIVE TISSUE CHARACTERIZATION SYSTEM AND METHOD” issued Feb. 13, 2007 with D. Geoffrey Vince, Anuja Nair and Jon D. Klingensmith as inventors, U.S. Pat. No. 7,215,802 entitled “SYSTEM AND METHOD FOR VASCULAR BORDER DETECTION” issued May 8, 2007 with Jon D. Klingensmith, Anuja Nair, Barry D. Kuban and D. Geoffrey Vince as inventors, U.S. Pat. No. 7,359,554 entitled “SYSTEM AND METHOD FOR IDENTIFYING A VASCULAR BORDER” issued Apr. 15, 2008 with Jon D. Klingensmith, D. Geoffrey Vince, Anuja Nair and Barry D. Kuban as inventors and U.S. Pat. No. 7,463,759 entitled “SYSTEM AND METHOD FOR VASCULAR BORDER DETECTION” issued Dec. 9, 2008 with Jon D. Klingensmith, Anuja Nair, Barry D. Kuban and D. Geoffrey Vince, as inventors, the teachings of which are hereby incorporated by reference herein in their entirety.

In order to perform the calculations, determinations, and logical operations disclosed herein, the automatic target and reference detection system may employ a number of variables with default values. However, the values of one or more of these variables may, if desired, be edited by the user to values other than the default value, in order to substitute, whether wholly or partially, the judgment of the user over the assumptions embedded in the automatic target and reference detection system. Some example variables are shown in Table 1.

TABLE 1
User-Modifiable Variables

| ID | description | range | unit | default | datatype | stepsize |
|---|---|---|---|---|---|---|
| APPSET 1 | Consecutive frames to meet criteria for | 0-100 | frames | 3 | whole number | 1 |
| | classification as target | | | | | |
| APPSET 2 | % Min Threshold of target criteria (wrt | 0-100 | 1% | 50 | whole number | 1 |
| | average) | | | | | |
| APPSET 3 | Primary criteria metric for target | Area, Min | text from list | Area | text | |
| | | Diameter, max | | | | |
| | | Diameter, Eff. | | | | |
| | | Diameter, Avg | | | | |
| | | Diameter | | | | |
| APPSET 4 | Stddev of Standard reference value in % | 0-100 | | 10 | whole number | 1 |
| APPSET 5 | Standard Reference value | See APPSET05 | | | | |
| | | details | | | | |
| APPSET 6 | Min Aspect Ratio value threshold for | 1-10 | None | 1.5 | fraction | 0.1 |
| | target | | | | | |
| APPSET 7 | Consecutive frames to meet criteria for | 0-100 | frames | 5 | whole number | 1 |
| | classification as reference | | | | | |
| APPSET 8 | Max Aspect Ratio value threshold for | 1-10 | | 1.5 | fraction | 0.1 |
| | reference( wrt to average) | | | | | |
| APPSET 9 | % Min Threshold of reference criteria | 0-100 | | 120 | whole number | 1 |
| APPSET 10 | Primary criteria metric for reference | Area, Min | | Area | text | |
| | | Diameter, max | | | | |
| | | Diameter, Eff. | | | | |
| | | Diameter, Avg | | | | |
| | | Diameter, | | | | |
| | | Aspect Ratio, | | | | |
| | | Flow Resistance | | | | |

APPSET 4 and APPSET 5 may be useful for identification of target and reference locations using standardized or “normal” anatomy from literature, but may not be useful for computing target and reference locations using the novel methods disclosed herein. Table 2 includes user-editable standard anatomical measurements that may be used along with APPSET 4 and APPSET 5, whose default values come from medical literature.

TABLE 2
User-Modifiable Example “Standard” Vein Dimensions

| | | | | de- | data- | step- |
|---|---|---|---|---|---|---|
| ID | description | range | unit | fault | type | size |
| APPSET5 A | CIV Standard | 0-300 mm2 | mm2 | 200 | fraction | any |
| | Area | | | | | |
| APPSET5 B | EIV Standard | 0-300 mm3 | mm2 | 150 | fraction | any |
| | Area | | | | | |
| APPSET5 C | CFV Standard | 0-300 mm4 | mm2 | 125 | fraction | any |
| | Area | | | | | |
| APPSET5 D | CIV Standard | 0-20 mm | mm | 16 | fraction | any |
| | Eff Dia | | | | | |
| APPSET5 E | EIV Standard | 0-20 mm | mm | 14 | fraction | any |
| | Eff Dia | | | | | |
| APPSET5 F | CFV Standard | 0-20 mm | mm | 12 | fraction | any |
| | Eff Dia | | | | | |

In an example, depending on the implementation and the selections made by the user, the intraluminal imaging display screen 500 can show reference and landing zones that are calculated based on lumen area, lumen minimum diameter, lumen effective diameter, other lumen dimensions (e.g., lumen aspect ratio, flow resistance, etc.), or a stenosis formula. In an example stenosis formula, such as:

$$\% area stenosis = ( normal area - lesion area ) / normal area \times 100 \qquad ( EQN . 1 )$$

In a clinically representative example, the threshold of % area stenosis for a lesion to be considered significant is approximately 54%. Another stenosis formula is:

$$\% diameter stenosis = \frac{\begin{matrix}( normal estimated diameter - \\ lesion minimum diameter )\end{matrix}}{normal mean diameter \times 100} \qquad ( EQN . 2 )$$

In a clinically representative example, the threshold of % diameter stenosis for a lesion to be considered significant is approximately 61%.

It is noted that the lumen dimension shown at bookmarked locations along the ILD 520 may be based on the variable APPSET 10, so that it defaults to showing the lumen area. However, other options selectable by the user may include, for example, effective diameter, minimum diameter, maximum diameter, aspect ratio, flow resistance, or other lumen metrics depending on the implementation.

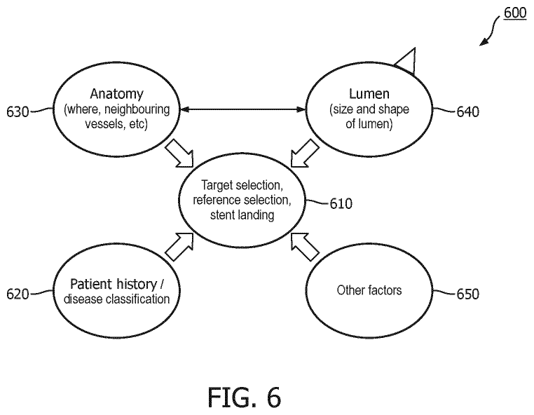

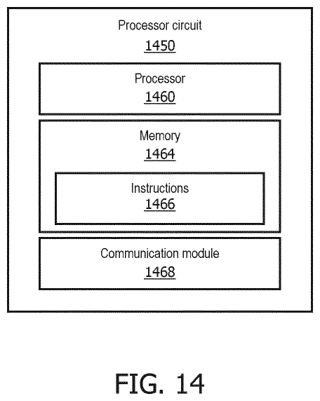

FIG. 6 is a schematic, diagrammatic representation of a target frame, reference frame, and stent landing zone selection method 600, in accordance with at least one embodiment of the present disclosure. With a goal of selection 610 of a target location, reference location, stent landing zone, or other regions of interest, the clinician must first consider the patient history 620 and the classification of vascular disease(s) the patient is known to have. The selection 610 of the target location may for example be determined by the processing system 106 (see FIG. 1) or processor circuit 1450 (see FIG. 14), as described below. The patient history 620 may for example be obtained from medical records stored in a memory accessible to the intraluminal imaging system. For example, if a vein segment is healthy or contains a focal lesion, then 0 or 1 target frames and 0 or 1 reference frames may be expected. However, if the segment includes a diffused lesion, then 1 reference frame may be expected, but with no identifiable target frame. If the segment includes excessive compression, then 1 target frame and 1 reference frame may be expected, e.g. in a prestenotic dilation. Thus, there may be zero, one, or multiple target and reference frames within a given segment.

Additionally, the method 600 may receive patient-specific anatomy 630, including branches or confluences, proximity of neighboring vessels, the location of a lesion, etc., along with the specific characteristics 640 of the lumen of interest, such as lumen area, flatness/roundness (e.g., aspect ratio), etc. Both the patient-specific anatomy 630 and the specific characteristics 640 may for example be obtained from extraluminal (e.g., x-ray, CT images) and/or intraluminal (IVUS) images.

Additionally, the method 600 consider other factors 650, including for example cost, time, the age and overall health of the patient, or reference values for blood vessel dimensions. In a clinically relevant example, anatomical normal “theoretical values” for area and diameter (which may vary up to +/−10%) include:
IVC: 250 mm², 18 mm
CIV: 200 mm², 16 mm
EIV: 150 mm², 14 mm
CFV: 125 mm², 12 mm

However, not all patients conform to these standardized dimensions, not all clinicians agree with them, and calculations based on these values do not always achieve an optimal result. In addition, clinicians may select IVUS image frames that are “most relevant” in their opinions, and that are not necessarily target or reference frames in the usual sense.

As a result of method 600, clinical judgments (which can vary widely, resulting in significant differences in the selected reference, target, and landing locations) can be augmented with an algorithmic, real-time process that is simple, easy to understand, easy to communicate, and produces repeatable results, so that the user can know exactly how the algorithm picked a target and reference frame in each segment.

In addition, the automatic target and reference detection system may allow users to override the default values of a number of variables, such that the logic operations described herein can be performed again using different assumptions. Thus, a clinician can examine, in real time, any changes in target or landing locations that occur when, for example, the lumen metric (e.g., the variable APPSET 3 or APPSET 10) is changed from “area” to “aspect ratio”, or when the threshold for the lumen metric (e.g., variable APPSET 2 or APPSET 9) is increased or decreased. This real-time tunability may allow experienced practitioners to determine robust solutions that improve the likelihood of successful treatment, with higher confidence in the results than would be possible using traditional methods, and in a time frame that would not be possible using traditional methods.

In an example, a vein may be considered compressed or otherwise diseased if it contains a location that has 53.6% of the area or 61.2% of the diameter of nearby healthy portions of the vein. In some instances, any portions of that vein with values equal to or lower than these may be considered a “target zone”, within which a specific target location or target frame can be selected, as described below. In other instances, a combination of values may be considered to determine the existence, location, and severity of a compression, lesion, or other disease. Thus, there may be multiple target candidates from which target zone is picked and s target frame identified. The algorithm employs reference values derived from IVUS measurements obtained during the pullback procedure (e.g., a reference frame identified in real time as described below). However, the automatic target and reference detection system may include a user-selectable setting to allow the clinician to compute target and reference frames based on standardized, theoretical anatomic averages obtained from published literature.

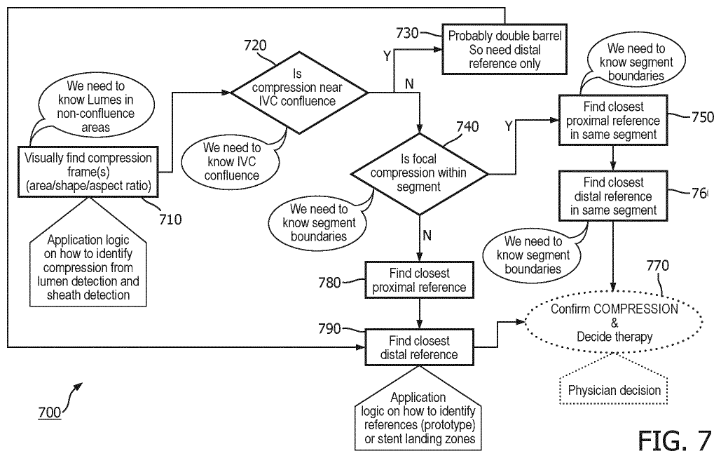

FIG. 7 is a diagrammatic representation, in flow diagram form, of an example pre-treatment target zone and reference frame detection method 700, in accordance with at least one embodiment of the present disclosure. It is understood that the steps of method 700 may be performed in a different order than shown in FIG. 7, additional steps can be provided before, during, and after the steps, and/or some of the steps described can be replaced or eliminated in other embodiments. One or more of steps of the method 700 can be carried by one or more devices and/or systems described herein, such as components of the intraluminal imaging system 100, PIM 194, processing system 106, and/or processor circuit 1450.

In step 710, the method 700 includes receiving a plurality of IVUS images (e.g., from an IVUS pullback), determining whether a compression or other disease is present in the IVUS images, and identifying specific IVUS image frames within the pullback sequence that include the compression or other disease. Such identification may be based on image recognition or other determination of the area, shape, or aspect ratio of the vessel lumen, or on other factors as described herein. The logic described herein may identify both the lumen and the sheath of the vessel in order to determine whether, and to what degree, compression or other disease is present in the vessel.

In step 720, the method 700 includes determining whether the detected compression or other disease is located proximate to (e.g., within a specified number of frames or millimeters of) the confluence of the inferior vena cava (IVC). If yes, execution proceeds to step 730. If no, execution proceeds to step 740.

In step 730, the method 700 includes determining that the reference frame should be located distal of, rather than proximal of, the compressed region. Execution then proceeds to step 790.

In step 740, the method 700 includes determining whether a compression exists within the current segment of the vessel, thus defining a target zone. If yes, execution proceeds to step 750. If no, execution proceeds to step 780.

In step 750, the method 700 includes identifying the most optimal reference frame that is proximal of the compression or other disease and located within the same vessel segment. Execution then proceeds to step 760.

In step 760, the method 700 includes identifying the most optimal reference frame that is distal of the compression or other disease and located within the same vessel segment. Execution then proceeds to step 770.

In step 770, the method 700 includes reporting the reference frame(s) (e.g., by displaying them on a display), so that a clinician can confirm the existence and location of the compression, and determine appropriate treatment. The method is now complete.

In step 780, the method 700 includes finding the most optimal reference frame that is located proximal of the compression or other disease, and not necessarily located within the current segment.

In step 790, the method 700 includes finding the most optimal reference frame that is located distal of the compression or other disease, and not necessarily located within the current segment.

In some cases, the proximal and distal reference frames may define a landing zone for stenting such that, for example, the proximal end of the stent is aligned with the proximal reference frame, and the distal end of the stent is aligned with the distal reference frame. However, clinical judgment on the part of the clinician may indicate alternative placement of the stent.

It is noted that the terms “target” and “reference” are merely exemplary, and that other terms may be used instead or in addition be used, including but not limited to: target frame, compression frame, frame of interest, treatment frame, reference frame, healthy frame, etc. Reference frames can be on either side or both sides of the compression. The automatic target and reference detection system may identify one or a plurality of reference frames per segment. The reference frame can be used for comparison to a target frame (e.g., evaluate extent of disease/compression, pre-treatment to inform clinical decision about treatment). The one or plurality of reference frames can be used and identified in a screen display as potential proximal/distal landing zone for stent. In other embodiments, the one or plurality of reference frames need not be used or identified as a potential landing zone for stent. The automatic target and reference detection system may identify one target frame and one reference frame per segment, or multiple target frames and/or multiple reference frames per segment.

It is noted that all flow diagrams disclosed herein are provided for exemplary purposes; a person of ordinary skill in the art will recognize myriad variations that nonetheless fall within the scope of the present disclosure. For example, the logic of flow diagrams may be shown as sequential. However, similar logic could be parallel, massively parallel, object oriented, real-time, event-driven, cellular automaton, or otherwise, while accomplishing the same or similar functions. In order to perform the methods described herein, a processor may divide each of the steps described herein into a plurality of machine instructions, and may execute these instructions at the rate of several hundred, several thousand, several million, or several billion per second, in a single processor or across a plurality of processors. Such rapid execution may be necessary in order to execute the method in real time or near-real time as described herein. For example, in order to provide precise, repeatable identification of target frames, reference frames, landing zones, etc., the system may perform the steps disclosed herein during an IVUS pullback procedure where shorter procedure times may be associated with improved patient outcomes. Thus, if the pre-treatment target and reference frame detection method 700 does not decrease the time required to perform the procedure and/or the accuracy of results, clinicians may be reluctant to adopt it.

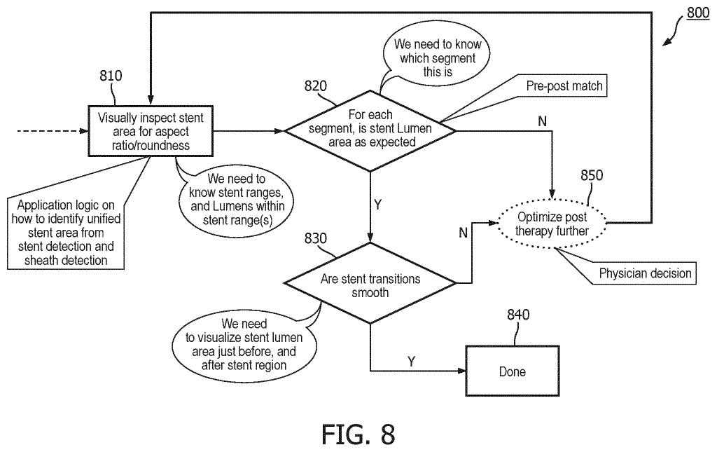

FIG. 8 is a diagrammatic representation, in flow diagram form, of an example post-treatment stent inspection method or minimum stent area computation method 800, in accordance with at least one embodiment of the present disclosure.

In step 810, the method 800 includes receiving a plurality of IVUS images of the stent (e.g., captured and stored during an IVUS pullback), identifying the proximal and distal ends of the stent in the images, and evaluating the aspect ratio (the radio between the largest diameter and the smallest diameter of an ellipse) to determine the roundness of the stent in cross-section. Other variables may be evaluated as well, such as lumen cross-sectional area, max. diameter, min. diameter, flow resistance, etc.

In step 820, the method 800 includes determining whether, for each stented segment of the vessel, the stent lumen area (or aspect ratio, etc.) is within expected parameters for successful treatment (e.g., whether it will support increased blood flow to the expected degree). If yes, execution proceeds to step 830. If no, execution proceeds to step 850.

In step 830, the method 800 includes determining whether the stent transitions are smooth. For example, if the diameter of the vessel is similar both inside and outside the stent, then the transitions may be acceptably smooth, and execution proceeds to step 850. If the diameter outside the stent is significantly different than thew diameter inside the stent, then the transitions may not be acceptably smooth, and execution proceeds to step 850.

In step 840, the method includes reporting the results of steps 810-820 (e.g., by displaying them on a display). The method 800 is complete.

In step 850, the method 800 includes determining that further therapy, or optimization of the existing therapy, according to the judgment of the clinician, is necessary.

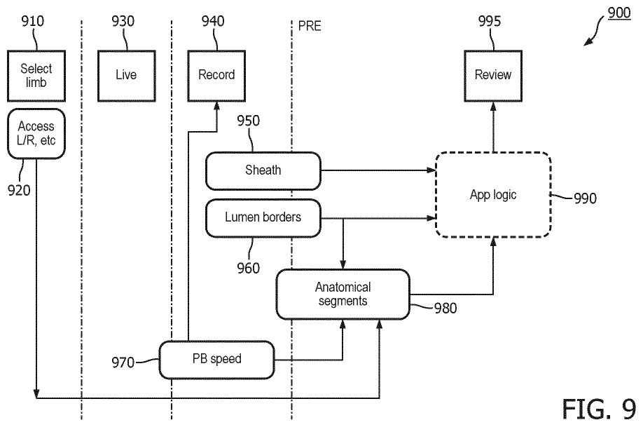

FIG. 9 is a diagrammatic representation, in block diagram form, of at least a portion of an example pre-treatment automatic target and reference detection system or method 900, in accordance with at least one embodiment of the present disclosure. It is understood that the steps, modules, inputs, or outputs of the method 900 may be occur or be performed in a different order than shown in FIG. 9, additional steps, modules, inputs, or outputs can be provided before, during, and after the steps, and/or some of the steps described can be replaced or eliminated in other embodiments. One or more of steps, modules, inputs, or outputs of the method 900 can be carried out by one or more devices and/or systems described herein, such as components of the intraluminal imaging system 100, PIM 194, processing system 106, and/or processor circuit 1450.

In step 910, the method 900 includes prompting the user to select which limb will be treated.

In step 920, the method 900 includes selecting which side of the body the limb is on (e.g., left or right), and which access point will be used to enter the target vessel in that limb.

In step 930, the method 900 includes prompting the user to switch on the IVUS system and insert the intraluminal imaging device into the target vessel of the patient and capturing live IVUS images of the interior of the vessel. The clinician may for example advance the imaging device past the compressed or otherwise diseased portion of the vessel and prepare to perform a pullback procedure.

In step 940, the method 900 includes accepting an input from the user to switch to a recording mode, wherein the IVUS images captured by the intraluminal imaging device such that, when a pullback procedure is performed, images of the diseased portions of the vessel, along with healthy portions surrounding the diseased portions, are stored for analysis. The system records images of the sheath 950 and vessel lumen borders 960, and calculates a pullback speed 970 by analyzing the images as they are recorded. The sheath may for example refer to the outermost portion of the imaging catheter. In some embodiments, the method may include measuring values relative to the sheath (e.g., distance between sheath and lumen border, area between sheath and lumen border, etc.).

Within the captured images, the system then identifies anatomical segments 980 of the vessel that the intravascular imaging probe passes through, based on the pullback speed 970, as well as per-frame measurement of the dimensions of, and image recognition of, the sheath 950 and lumen borders 960, as well as derived quantities thereof. The per-frame measurements and derived quantities may for example include each of the possible lumen metrics that can be specified by [APPSET 3] and [APPSET 10].

In step or module 990, application logic receives the images and per-frame measurements of the sheath 950 and lumen borders 960, as well as the pullback speed 970 and the identified anatomical segments 980. The application logic then computes target and reference locations within the vessel.

In step 995, the method 900 includes displaying or otherwise presenting the target location(s), reference location(s), IVUS images, anatomical segments, and per-frame measurements to the clinician for review.

The application logic described in step 990 may for example perform the following steps:
990 A: Identify Vessel Segment and exclude confluences
990 B: In each segment, excluding no go regions (confluences) that cannot be used as target or reference locations.
990 C: Between the start of each segment and the start of the next branch or confluence, exclude the last 15 frames of the caudal window. For example, the CIV Segment may be defined as CIV Start to EIV Start−15. Similarly, EIV Segment=EIV Start to CFV Start or Sheath Start−15, and CFV Segment=CFV Start to Sheath Start−15. Another example implementation may exclude a specified percentage (e.g., 5%, or a user-selectable value) of both the start and the end of each segment.
990 D: Find the average lumen metric of the segment, where the lumen metric has been specified by the user (e.g., through variables APPSET 3 and/or APPSET 10). Each frame of the segment may be analyzed such that the lumen metric is measured and stored, and an average is then taken of the stored values. Optionally, the method may filter out single-frame outliers (e.g., using a median filter). For example, the method may compute a histogram of lumen metric, and remove the top 25% and bottom 25% of the curve before computing the average. The system may then also compute a standard deviation of the remaining data points.

### Reference Frame

990 E: Identify a reference zone that meets the following criteria: Center of a window of [APPSET 7] frames where at least [APPSET 7] contiguous frames meet a primary condition (e.g., aspect ratio <1.5), a secondary condition (e.g., lumen metric >100% of average for the segment), and/or optionally a tertiary condition (e.g., max. and min. values of the lumen metric), as defined by the variables APPSET 6-APPSET 9. The start frame can be defined as the location where all specified conditions pass, and the stop frame can be defined as the location where at least one of the specified conditions fails. It is noted that there can be multiple reference zones in a segment, even if the method will eventually only show one reference frame to the user. It is further noted that the location of the reference frame is user-editable.

The aspect ratio can be maximum lumen diameter divided by minimum lumen diameter. Use of aspect ratio is particularly relevant for peripheral venous disease because aspect ratio is a measure of compression (e.g., how much another anatomy presses against the peripheral vein and blocks blood flow in the peripheral). Aspect ratio is typically not used to evaluate coronary arterial disease because compression is less of an issue in coronary arteries. Rather, plaque burden is used to evaluate blood flow blockages in coronary arteries because plaque buildup typically blocks blood flow in coronary arteries. In some embodiments, aspects of the present disclosure do not utilize plaque burden to evaluate the blood flow blockage, and instead utilize aspect ratio, which is a better metric for peripheral venous vasculature disease (e.g., compression). An aspect ratio of 1 (e.g., vein cross-section shaped like a circle) would be considered healthy/normal. Aspect ratios larger than 1 indicate that the vein cross-section shaped like an ellipse and are affected by compression. That is, another anatomy is pressing against the vein, changing the cross-sectional shape from a circle to an ellipse.
990 F: Identify a reference frame within the reference zone(s). In an example, the criterion for a reference frame is the frame within the reference zone(s) that has the maximum lumen metric. In case of multiple frames that may meet this criterion, the most caudal of these frames is selected. Alternatively, the reference frame may be selected as the most caudal single frame of at least [APPSET 7] consecutive frames in the reference zone(s).

### Target Frame

990 G: Identify the target zone, which includes all frames for which the following criteria are met: the center of a window of [APPSET1] frames where at least [APPSET1] contiguous frames are less than the specified threshold for the lumen metric (e.g., APPSET 2 or APPSET 3). For example, the threshold may default to 50% for an area metric, or 62% for a diameter metric. A secondary metric may optionally be specified. The start frame of the target zone is where both conditions pass (e.g., frame contiguity condition, or threshold condition). The stop frame of the target zone is where at least one condition fails. It is notes that there can be multiple target zones in a segment, even if a single target frame is eventually selected. All of the identified target zones may be shown to the user (e.g., on the ILD or roadmap images, as shown for example in FIG. 5). It is further noted that the location of the target frame is user-editable.
990 H: Identify the target frame, which is the frame within the target zone(s) that has the minimum lumen metric. In case of multiple frames that may meet this criterion, the most cranial frame is selected.
990 I: Calculate and display a difference metric associated with the target frame. The difference metric may be calculated based on a stenosis formula such as:

$$\% Difference Area : ( reference area - target area ) / reference area \times 100 \qquad ( EQN . 3 )$$

$$\% Difference Diameter : \frac{( reference mean diameter - target minimum diameter )}{reference mean diameter \times 100} \qquad ( EQN . 4 )$$

As an alternative to steps 990A-990I (hereinafter referred to as the complex calculations), the application logic 990 may optionally compute the reference and target frames by a simpler method, as follows. For each segment, find the frame that has the maximum value of the lumen metric [APPSET 3] and the frame that has the minimum the lumen metric [APPSET 3]. The minimum frame is identified as the target, and the maximum frame is identified as the reference.

In some embodiments, the user can switch between the complex and simplified calculations to see whether their results are meaningfully different. Furthermore, in both the complex and simplified calculations, the user can change [APPSET 3] to switch the lumen metric between values such as “Area”, “Min Diameter”, “Effective Diameter”, “Flow Resistance”, etc., to determine whether different lumen metrics result in meaningfully different locations for the reference and target frames. Thus, the clinician can “play around” with the settings in order to gain confidence that the results are correct and robust.

It is noted that flow resistance can be expressed in area (Ae) and aspect ratio (AR). Thus, a diameter, area, aspect ratio, etc. of the vessel at a given location is related to the flow or flow resistance at that location. Resistance scales inverse quadratically with area, and roughly linear with aspect ratio, as shown below:

$$Re = 4 η L \frac{( {a}^{2} + {b}^{2} )}{π {a}^{3} {b}^{3}} = \frac{4 η L}{Ae} \frac{( {a}^{2} + {b}^{2} )}{{a}^{2} {b}^{2}} = \frac{4 η L}{Ae} ( \frac{1}{{b}^{2}} + \frac{1}{{a}^{2}} ) \qquad EQN . 5$$

$$Re = \frac{4 η L}{Ae} ( \frac{π}{\frac{Ae}{AR}} + \frac{π}{Ae AR} ) = \frac{π 4 η L}{{Ae}^{2}} ( \frac{1}{\frac{1}{AR}} + \frac{1}{AR} ) \qquad EQN . 6$$

$$Re = \frac{π 4 η L}{{Ae}^{2}} ( AR + \frac{1}{AR} ) \qquad EQN . 7$$

$$Ae = pi a b \qquad EQN . 8$$

$$AR = a / b \qquad EQN . 9$$

$$Ae * AR = pi a b * a / b = pi a \hat{} 2 \qquad EQN . 10$$

$$Ae / AR = pi a b / ( a / b ) = pi b \hat{} 2 \qquad EQN . 11$$

Steps similar to those described above can be used for determination of post-stenotic dilation, e.g., the change in lumen profile at caudal end of the pullback. If compression detected is more than 60% of criteria, then the method may exclude frames that are cranial to the caudal edge of the segment.

As yet another alternative, the target zone may be computed using theoretical “normal” values from published literature, as follows: the target zone includes at least [APPSET 1] consecutive frames that meet a primary condition of [APPSET2] compression of a theoretical value [APPSET 5a-APPSET 5f] of a lumen metric [APPSET 3], within a specified range [APPSET 4]. In some embodiments, a secondary criterion may also be selected to be checked along with the primary condition. The secondary condition may become primary condition if the primary condition does not apply to the current pullback. For example, aspect ratio is used implicitly in the computation of flow, as described below.

This option allows a clinician to revert to alternative target zone calculations in order to verify whether this yields a different result than either the complex or simplified calculations. The following equations may be used to determine values such as area and/or flow resistance R using diameter, lumen radius r (e.g., average, such as mean, median, or mode), minimum lumen radius a, and maximum lumen radius b, viscosity η, length L of the volume through which flow is considered, and flow. Any one or more of the values (e.g., measured, calculated, derived, etc.) can be lumen metrics.

$${A}_{c} = π {r}^{2} \qquad EQN . 8$$

$${\underline{R}}_{\underline{c}} = 8 η L / π {r}^{4} \qquad EQN . 9$$

$$= 8 π η L / {A}_{c}^{2} ∼ η L / {A}^{2} \qquad EQN . 10$$

$${A}_{3} = π ab \qquad EQN . 11$$

$${R}_{e} = 4 η L ( {a}^{2} + {b}^{2} ) / π {a}^{3} {b}^{3} \qquad EQN . 12$$

$$= 4 π η L ( π {a}^{2} + π {b}^{2} ) / {A}_{e}^{3} \qquad EQN . 13$$

$$\begin{matrix}= 8 π η L ( {A}_{ca} + {\underline{A}}_{\underline{cb}} ) / 2 / {A}_{e}^{3} & ∼ η L \hat{A} / {A}^{3} ∼ η L / {A}^{2}\end{matrix} \qquad EQN . 14$$

Area can be computed by pixel counting or through other geometric techniques, and a diameter is derived from this. Diameter can be found from this area or by finding the distance between opposite points on the contour passing through the centroid. Similarly flow resistance can be computed from area. The lumen metric(s) may be chosen by the user through settings.

In some embodiments, one or more stent landing frames may be selected based on certain characteristics of the identified reference and target zones. For example, in the case of a target appearing at the IVC confluence, the closest non-target frame may be a cranial stent landing frame. A caudal stent landing frame is one which may, for example, be the closest reference frame caudal to the target zone, excluding branches. Other examples include finding the single stent landing frame at each end of a region that encompasses multiple target and reference zones, either within a segment or across a boundary between two segments. User-configurable thresholds can be used to define how large the gap can be between zones, in order to consider it one stent zone. These stent landing frames can be editable by the user as well.

All block diagrams disclosed herein are provided herein for exemplary purposes; a person of ordinary skill in the art will recognize myriad variations that nonetheless fall within the scope of the present disclosure. For example, block diagrams may show a particular arrangement of components, modules, services, steps, processes, or layers, resulting in a particular data flow. It is understood that some embodiments of the systems disclosed herein may include additional components, that some components shown may be absent from some embodiments, and that the arrangement of components may be different than shown, resulting in different data flows while still performing the methods described herein.

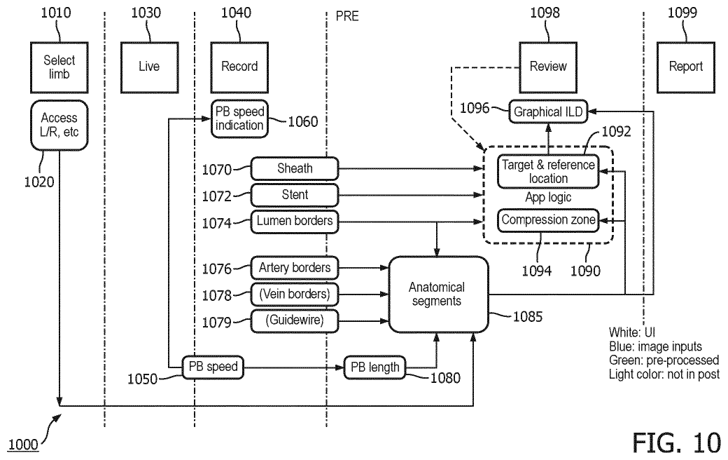

FIG. 10 is a diagrammatic representation, in block diagram form, of at least a portion of an example pre-treatment automatic target and reference detection system or method 1000, in accordance with at least one embodiment of the present disclosure. The method 1000 may in some cases be similar to the method 900, but may involve a larger number of measurements of the lumen and its surroundings.

In step 1010, the method 1000 includes prompting the user to select which limb will be treated.

In step 1020, the method 1000 includes selecting which side of the body the limb is on (e.g., left or right), and which access point will be used to enter the target vessel in that limb.

In step 1030, the method 1000 includes prompting the user to switch on the IVUS system and insert the intraluminal imaging device into the target vessel of the patient and capturing live IVUS images of the interior of the vessel. The clinician may for example advance the imaging device past the compressed or otherwise diseased portion of the vessel and prepare to perform a pullback procedure.

In step 1040, the method 1000 includes accepting an input from the user to switch to a recording mode, wherein the IVUS images captured by the intraluminal imaging device such that, when a pullback procedure is performed, images of the diseased portions of the vessel, along with healthy portions surrounding the diseased portions, can be stored for analysis. The system records images of the sheath 1070 and vessel lumen borders 1074, and calculates a pullback speed 1050 by analyzing the images as they are recorded. In addition, the system identifies and measures artery borders 1076 and vein borders 1078 of any neighboring blood vessels to the vessel of interest, as well as the guidewire or catheter borders 1079, if a guidewire or catheter is present in the IVUS images.

The system then identifies anatomical segments 1085 of the vessel that the intravascular imaging probe passes through, based on the pullback speed 1050, pullback length 1080, as well as per-frame measurement of the dimensions of, and image recognition of, the sheath 1070, stent 1072 (if present), lumen borders 1074, artery borders 1076, vein borders 1078, and guidewire or catheter borders 1079, as well as derived quantities thereof. The per-frame measurements and derived quantities may for example include each of the possible lumen metrics that can be specified by [APPSET 3] and [APPSET 10].

In step or module 1090, application logic receives the images and per-frame measurements of the sheath 1070 and lumen borders 1060, as well as the pullback speed 1050 and the identified anatomical segments 1085. The application logic then computes the compression zone 1094 (including both the target and reference zones as described above), as well as the target and reference locations 1092 locations within the vessel, according to one or more of the methods described above for FIG. 9, or related methods that produce the same or similar results.

In step 1096, the method 1000 includes updating and/or annotating a graphical ILD and/or roadmap display (as shown for example in FIG. 5) to include at least one of the target location(s), reference location(s), IVUS images, anatomical segments, and per-frame measurements for review by the clinician.

In step 1098, the method 1000 includes prompting the clinician to review the ILD and/or roadmap display, and/or waiting for additional inputs while the clinician reviews the displayed data. Such inputs may for example include commands to change the lumen metric and/or the method of calculating the reference and target frames. If such input is received, execution returns to step or module 1090.

In step 1099, the method includes generating a report that includes at least one of the target location(s), reference location(s), IVUS images, anatomical segments, and per-frame measurements. The method is now complete.

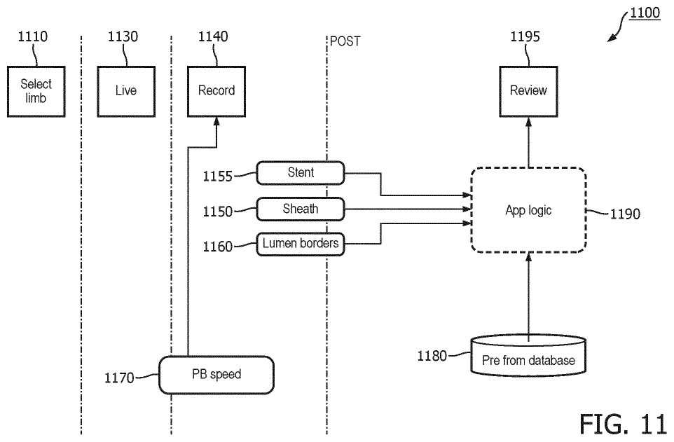

FIG. 11 is a diagrammatic representation, in block diagram form, of at least a portion of an example post-treatment automatic stent evaluation system or method 1100, in accordance with at least one embodiment of the present disclosure.

In step 1110, the method 1100 includes prompting the user to select which limb will be treated.

In step 1130, the method 1100 includes prompting the user to reinsert the intraluminal imaging device into the target vessel of the patient, and again capturing live IVUS images of the interior of the vessel. The clinician may for example advance the imaging device past the stented or otherwise treated portion of the vessel and prepare to perform a pullback procedure.

In step 1140, the method 1100 includes accepting an input from the user to switch to a recording mode, wherein the IVUS images captured by the intraluminal imaging device such that, when a pullback procedure is performed, images of the stented portions of the vessel, along with healthy portions surrounding the stented portions, can be stored for analysis. The system records images of the stent 1155, sheath 1150, and vessel lumen borders 1160, and calculates a pullback speed 1170 by analyzing the images as they are recorded.

In step or module 1190, application logic receives the images and per-frame measurements of the sheath 1150 and lumen borders 1160, as well as the pullback speed 1170. The application logic also receives the pre-treatment data 1180, including images and calculations from the pre-treatment analysis (see FIGS. 9 and 10), which have been stored in a database. The application logic then evaluates the stent as follows:
1190 A: Identify the proximal and distal ends of the stent. It is noted that the locations of the proximal and distal stent edges are user-editable.
1190 B: Confirm that at least 5 consecutive frames conform to the expected dimensions of the stent.
1190 C: Identify the stent's maximum constriction, e.g., the frame within the stent that exhibits the least desirable lumen metric (e.g., minimum stent area or MSA). It is noted that the location of the most constricted frame is user-editable.
1190 D: Compute the aspect ratio (AR) of the most constricted frame, if different from the lumen metric. It is noted that both the lumen metric and the AR may be clinically important in evaluating whether the stent has deployed successfully and has opened the vessel sufficiently to permit healthy blood flow.

In step 1195, the method 1100 includes displaying or otherwise presenting the location, lumen metric and aspect ratio of the most constricted frame, IVUS images, stent proximal and distal ends, and per-frame measurements for review by the clinician. The system may then wait for additional inputs while the clinician reviews the displayed data. Such inputs may for example include commands to change the lumen metric (e.g., minimum stent area, minimum effective diameter, maximum flow resistance, etc.). If such input is received, execution returns to step or module 1190. Based on the presented data, the clinician may opt for further treatment such as further expansion of the stent, placement of additional stents, or other treatments as appropriate.

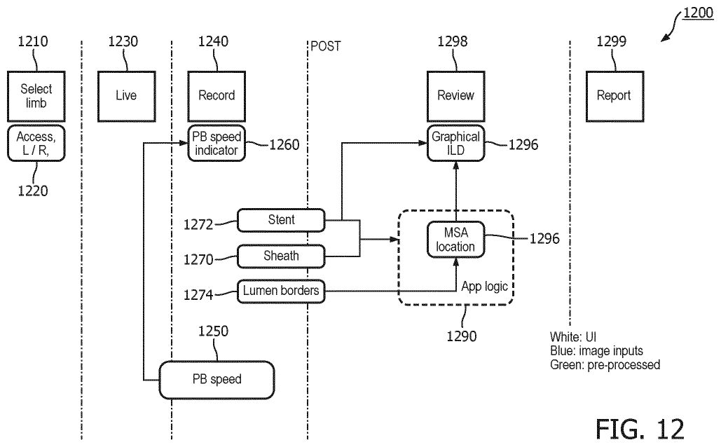

FIG. 12 is a diagrammatic representation, in block diagram form, of at least a portion of an example post-treatment automatic stent evaluation system or method 1200, in accordance with at least one embodiment of the present disclosure.

In step 1210, the method 1200 includes prompting the user to select which limb will be treated.

In step 1220, the method 1200 includes selecting which side of the body the limb is on (e.g., left or right), and which access point will be used to enter the target vessel in that limb.

In step 1230, the method 1200 includes prompting the user to reinsert the intraluminal imaging device into the target vessel of the patient, and again capturing live IVUS images of the interior of the vessel. The clinician may for example advance the imaging device past the stented or otherwise treated portion of the vessel and prepare to perform a pullback procedure.

In step 1240, the method 1200 includes accepting an input from the user to switch to a recording mode, wherein the IVUS images captured by the intraluminal imaging device such that, when a pullback procedure is performed, images of the stented portions of the vessel, along with healthy portions surrounding the stented portions, can be stored for analysis. The system records images of the stent 1272, sheath 1270, and vessel lumen borders 1274, and calculates a pullback speed 1250 by analyzing the images as they are recorded. In some embodiments, the pullback speed 1250 may be displayed in real time on a pullback speed indicator 1260 that is visible to the clinician during the pullback.

In step or module 1290, application logic receives the images and per-frame measurements of the sheath 1270 and lumen borders 1274, as well as the pullback speed 1250. The application logic then evaluates the stent as follows:
1290 A: Identify the proximal and distal edges of the stent. It is noted that the locations of the proximal and distal stent edges are user-editable.
1290 C: Identify the frame within the stent that exhibits the minimum stent area (MSA). It is noted that the location of the MSA frame is user-editable.
1290 D: Compute the aspect ratio (AR) of the MSA frame. It is noted that both the MSA and the AR may be clinically important in evaluating whether the stent has deployed successfully and has opened the vessel sufficiently to permit healthy blood flow.

In step 1296, the method 1200 includes updating and/or annotating a graphical ILD and/or roadmap display (as shown for example in FIG. 5) to include at least one of the MSA location, aspect ratio of the MSA location, IVUS images, stent proximal and distal edges, and per-frame measurements for review by the clinician, as well as prompting the clinician to review the ILD and/or roadmap display.

In step 1298, the method 1200 includes waiting for additional inputs while the clinician reviews the displayed data. Such inputs may for example include commands to change the lumen metric (e.g., other variables than minimum stent area, such as minimum effective diameter, maximum flow resistance, etc.). If such input is received, execution returns to step or module 1290. Based on the presented data, the clinician may opt for further treatment such as further expansion of the stent, placement of additional stents, or other treatments as appropriate.

In step 1299, the method includes generating a report that includes at least one of the MSA location, aspect ratio of the MSA location, IVUS images, stent proximal and distal edges, and per-frame measurements. The method is now complete.

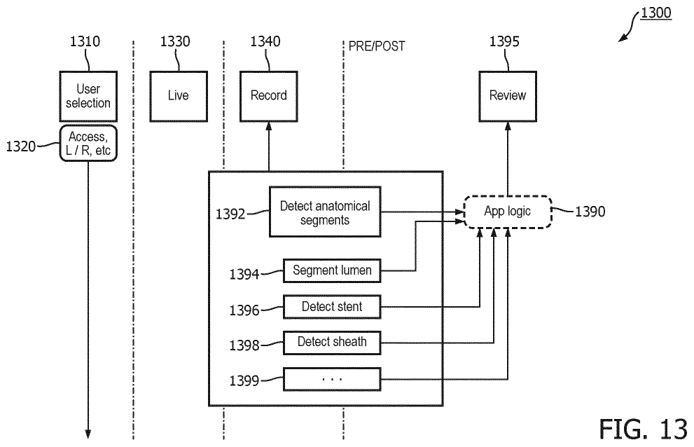

FIG. 13 is a diagrammatic representation, in block diagram form, of at least a portion of an example pre- and post-treatment automatic target identification, reference identification, and stent evaluation system or method 1300, in accordance with at least one embodiment of the present disclosure.

In step 1310, the method 1300 includes prompting the user to select which limb will be treated.

In step 1330, the method 1300 includes prompting the user to insert the intraluminal imaging device into the target vessel of the patient, and then capturing live IVUS images of the interior of the vessel. The clinician may for example advance the imaging device past the region of interest within the vessel and prepare to perform a pullback procedure.

In step 1340, the method 1300 includes accepting an input from the user to switch to a recording mode, and then storing the IVUS images captured the intraluminal imaging device such that, when a pullback procedure is performed, images of the vessel's region of interest, along with healthy portions surrounding the region of interest, can be analyzed.

The captured IVUS images from the pullback are then analyzed, in real time, by a detecting anatomical segments 1392, segmenting the lumen 1394, detecting stent 1396, detecting a sheath 1398, and/or other image analysis 1399. Aspects of modules/steps 1392, 1394, 1396, 1398, and/or 1399 can be similar to those described in the patent applications, patents, and/or publications mentioned herein. For example, modules/steps 1392, 1394, 1396, 1398, and/or 1399 can be autogenerated using techniques bed in the patent applications, patents, and/or publications mentioned herein. In some instances, modules/steps 1392, 1394, 1396, 1398, and/or 1399 come from manually drawn inputs and need not be autogenerated. modules/steps 1392, 1394, 1396, 1398, and/or 1399 can also using image processing, machine learning, or deep learning. In some instances, one or more modules/steps 1392, 1394, 1396, 1398, and/or 1399 is performed only after another one or more of modules/steps 1392, 1394, 1396, 1398, and/or 1399. For example, one or more modules/steps 1392, 1394, 1396, 1398, and/or 1399 uses the output of another one or more of modules/steps 1392, 1394, 1396, 1398, and/or 1399.

In an example, modules/steps 1392, 1394, 1396, 1398, and/or 1399 are machine learning (ML) algorithms that have been trained on annotations from experts, using data from multiple sites and subpopulations. Modules/steps 1392, 1394, 1396, 1398, and/or 1399 may also derive training from benchtop experiments (e.g., pullback speed measurement experiments), animal experiments, etc. Modules/steps 1392, 1394, 1396, 1398, and/or 1399 are selected such that their accuracy falls within the normal variability of clinician judgments about lumen measurements, derived quantities, and other clinical decisions. Thus, modules/steps 1392, 1394, 1396, 1398, and/or 1399 may be, statistically speaking, at least as accurate as the population of clinicians using the system. Acceptability of the modules/steps 1392, 1394, 1396, and/or 1398 may be tested through user evaluations. In some embodiments, self-training of modules/steps 1392, 1394, 1396, 1398, and/or 1399 is ongoing, based on usage of the system in a clinical setting. Outputs of the modules/steps 1392, 1394, 1396, 1398, and/or 1399 may for example be reference zones, reference frames, target zones, target frames, per-frame measurements, derived quantities, stent evaluations, or otherwise.

In step or module 1390, application logic receives the outputs of modules/steps 1392, 1394, 1396, 1398, and/or 1399. The application logic may for example provide targets frames, reference frames, and stent evaluations based on the outputs. For example, where the outputs are per-frame measurements or derived quantities, the application logic may process this information according to any of the methods described above. Where outputs are reference frames, target frames, or stent evaluations, the application logic may combine the outputs of different modules/steps through a weighting process, where weights are determined theoretically, empirically, based on expert opinion, or combinations thereof. Dissimilar output types may be combined through a Kalman filter or other statistical process. Such application logic can be straightforward, and can produce fast, repeatable results. The application logic 1390 automatically determines at least one of the target location, the reference location, the proximal end of the stent, the distal end of the stent, or the location within the stent where the stent is most constricted.

In step 1395, the method 1300 includes displaying or otherwise presenting at least one of the target frame, reference frame, IVUS images, anatomical segments, and per-frame measurements (or in the case of a stent, the location, lumen metric and aspect ratio of the most constricted frame, and stent proximal and distal ends) for review by the clinician. The system may then wait for additional inputs while the clinician reviews the displayed data. Such inputs may for example include commands to change the lumen metric or calculation method. If such input is received, execution returns to step or module 1390. Otherwise, the method is complete.

FIG. 14 is a schematic diagram of a processor circuit 1450, according to embodiments of the present disclosure. The processor circuit 1450 may be implemented in the system 100, or other devices or workstations (e.g., third-party workstations, network routers, etc.), or on a cloud processor or other remote processing unit, as necessary to implement the method. As shown, the processor circuit 1450 may include a processor 1460, a memory 1464, and a communication module 1468. These elements may be in direct or indirect communication with each other, for example via one or more buses.

The processor 1460 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 1460 may also comprise another hardware device, a firmware device, or any combination thereof configured to perform the operations described herein. The processor 1460 may also be implemented as a combination of computing devices, e.g., a combination of a DSP and a microprocessor, a plurality of microprocessors, one or more microprocessors in conjunction with a DSP core, or any other such configuration.

The memory 1464 may include a cache memory (e.g., a cache memory of the processor 1460), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1464 includes a non-transitory computer-readable medium. The memory 1464 may store instructions 1466. The instructions 1466 may include instructions that, when executed by the processor 1460, cause the processor 1460 to perform the operations described herein. Instructions 1466 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 1468 can include any electronic circuitry and/or logic circuitry to facilitate direct or indirect communication of data between the processor circuit 1450, and other processors or devices. In that regard, the communication module 1468 can be an input/output (I/O) device. In some instances, the communication module 1468 facilitates direct or indirect communication between various elements of the processor circuit 1450 and/or the system 100. The communication module 1468 may communicate within the processor circuit 1450 through numerous methods or protocols. Serial communication protocols may include but are not limited to United States Serial Protocol Interface (US SPI), Inter-Integrated Circuit (I²C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the intraluminal imaging device) may be accomplished using any suitable wireless or wired communication technology, such as a cable interface such as a universal serial bus (USB), micro USB, Lightning, or FireWire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

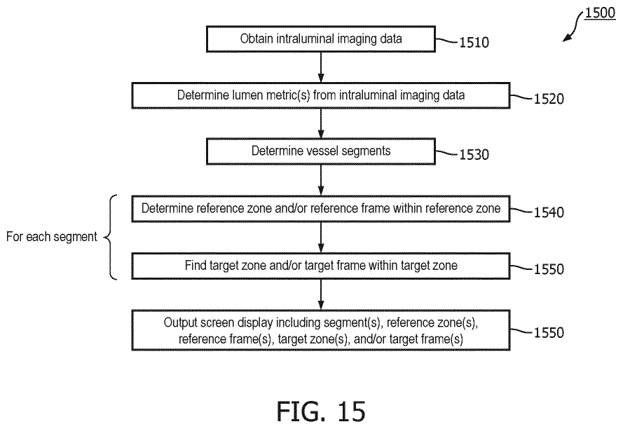

FIG. 15 is a diagrammatic representation, in flow diagram form, of an example pre-treatment target zone and reference frame detection method 1500, in accordance with at least one embodiment of the present disclosure.

In step 1510, the method includes obtaining the intraluminal image data, which may for example be a plurality of IVUS images stored during a pullback procedure.

In step 1520, the method includes automatically determining one or more lumen metrics, using the intraluminal imaging data, without user input to measure or verify the lumen metric.

In step 1530, the method includes automatically identifying the vessel segments, using the intraluminal imaging data, without user input to locate or verify the vessel segments.

In step 1540, the method includes automatically determining one or more reference zones and/or one or more reference frames within the reference zone(s), from the intraluminal imaging data, without user input to locate or confirm the reference zone and/or reference frame. This determination can be made:
(a) Using only lumen metrics from intraluminal imaging data,
(b) Using max lumen metric from intraluminal imaging data, or
(c) Using lumen metrics from intraluminal imaging data and lumen metrics from clinical/literature.

In step 1550, the method includes automatically determining one or more target zones and/or one or more target frames within the target zone(s), without user input to locate or confirm the target zone and/or target frame. This determination can be made:
(a) Using only lumen metrics from intraluminal imaging data
(b) Using max lumen metric from intraluminal imaging data
(c) Using lumen metrics from intraluminal imaging data and lumen metrics from clinical/literature.

Steps 1540 and 1550 are performed for each segment until all segments have been analyzed. Execution then proceeds to step 1560.

In step 1560, the method includes generating an output to a display screen that includes at least one of the segment(s), reference zone(s), reference frame(s), target zone(s), and/or target frame(s). The method is now complete.

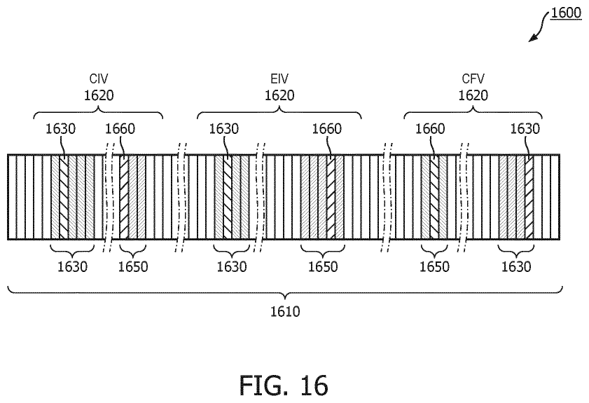

FIG. 16 is a schematic, diagrammatic representation of the analyzed IVUS image data 1600, in accordance with at least one embodiment of the present disclosure. The analyzed IVUS image data includes a plurality of cross-sectional image frames 1610 of the blood vessel, which have (by the methods described above) been divided into a plurality of segments 1620. Each segment may or may not include a reference zone 1630, which includes a reference frame 1640. Similarly, each segment may or may not include a target zone 1650, which includes a target frame 1660.

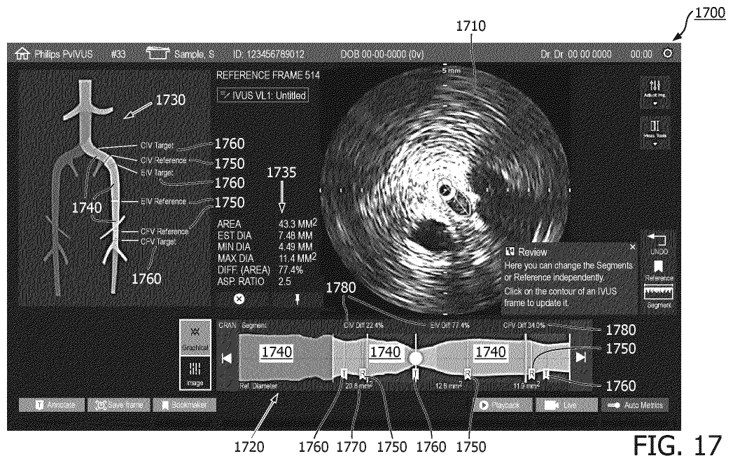

FIG. 17 illustrates an example intraluminal imaging display screen 1700, in accordance with at least one embodiment of the present disclosure. In this example, the screen display 1700 includes a current tomographic IVUS image 1710 from a series of successive tomographic images, an Image Longitudinal Display (ILD) 1720, and a graphical roadmap 1730. The ILD 1720 can be an image-based ILD or a graphical/stylized ILD. The graphical roadmap 1730 can be a cartoon or illustration of representative anatomy (e.g., peripheral venous vasculature), as opposed to an actual image(s) of a patient's anatomy. In some instances, the graphical roadmap can include actual image(s) of the patient's anatomy, such as x-ray, computed tomography (CT), ultrasound, and/or (magnetic resonance imaging) MRI images. A lumen metric window 1735 displays various lumen metrics of the current image 1710.

In the example shown in FIG. 1700, the segments 1740 are highlighted and labeled in both the ILD 1720 and the roadmap 1730. Also marked on the ILD 1720 and roadmap 1730 are the reference frames 1750 and the target frames 1760, along with respective values 1770 of the selected lumen metric at these locations. For example, in FIG. 17, the values 1770 are the lumen diameters from the reference frames in each of the different segments. Marked on the ILD are expressions or quantities 1780 that compare the magnitude of the selected lumen metric at the reference and target frames for each segment 1740. The width of the ILD 1720 varies with location, representing the magnitude of selected lumen metric in each image frame of the IVUS image data. It is noted that the target frames 1760 are generally proximate to the narrowest portion of each segment 1740, although this may not always be the case. Similarly, the reference frames 1750 are generally proximate to the widest portion of each segment, although this may not always be the case.

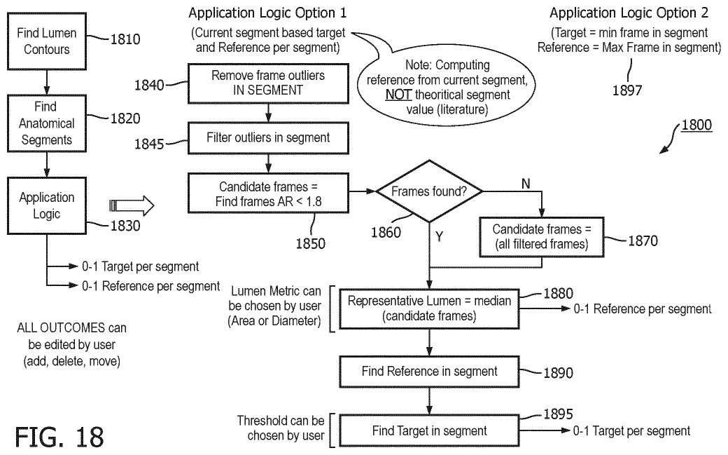

FIG. 18 is a diagrammatic representation, in flow diagram form, of an example pre-treatment target zone and reference frame detection method 1800, in accordance with at least one embodiment of the present disclosure.

In step 1810, the method includes receiving the IVUS images of the vessel and finding the lumen contours and lumen metrics in each frame.

In step 1820, the method includes identifying the anatomical segments of the vessel based on the lumen contours and lumen metrics.

In step 1830, the method includes initiating the application logic, as follows:

In step 1840, the method includes selectively removing frames from each segment whose lumen metrics and/or lumen contours are statistical outliers.

In step 1845, the method includes filtering outliers from the remaining distribution.

In step 1850, the method includes identifying candidate frames, where the candidates are frames with an aspect ratio less than 1.8, or frames that meet a different user-selectable criterion.

In step 1860, the method includes determining whether candidate frames were found. If no, execution proceeds to step 1870. If yes, execution proceeds to step 1880.

In step 1870, the method includes defining all of the filtered frames as candidate frames.

In step 1880, the method includes identifying median values for one or more lumen metrics among the candidate frames, or a user-selected lumen metric.

In step 1890, the method includes finding 0-1 reference frames within the segment.

In step 1895, the method includes finding 0-1 target frames within the segment.

An alternative to steps 1840-1895 for the application logic (step 1830) includes step 1897. In step 1897, the intraluminal image frame with the minimum lumen metric is identified as the target frame and the intraluminal image frame with the maximum lumen metric is identified as the references.

The method includes reporting the reference and target frames to the user (e.g., by identifying them on a screen display as shown for example in FIG. 17).

A number of variations are possible on the examples and embodiments described above. For example, other lumen metrics and other calculation methods could be used than those described herein, without departing from the spirit of the present disclosure. The system can include any logic that computes and displays target frames, reference frames, stent landing zones, and bookmarks in pre-treatment evaluation, and displays the MSA frame and other bookmarks in post-treatment evaluation. The present application is directed primarily toward peripheral venous and deep venous applications, but the same or similar logic could be applied to the examination of other body lumens.

The logical operations making up the embodiments of the technology described herein are referred to variously as operations, steps, objects, elements, components, or modules. Furthermore, it should be understood that these may occur or be arranged or performed in any order, unless explicitly claimed otherwise or a specific order is inherently necessitated by the claim language. All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of the automatic target and reference detection system. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” Unless otherwise noted in the claims, stated values shall be interpreted as illustrative only and shall not be taken to be limiting.

The above specification, examples and data provide a complete description of the structure and use of exemplary embodiments of the automatic target and reference detection system as defined in the claims. Although various embodiments of the claimed subject matter have been described above with a certain degree of particularity, or with reference to one or more individual embodiments, those skilled in the art could make numerous alterations to the disclosed embodiments without departing from the spirit or scope of the claimed subject matter. Still other embodiments are contemplated. It is intended that all matter contained in the above description and shown in the accompanying drawings shall be interpreted as illustrative only of particular embodiments and not limiting. Changes in detail or structure may be made without departing from the basic elements of the subject matter as defined in the following claims.

What is claimed is:

1. An intraluminal ultrasound imaging system, comprising:
a processor circuit configured for communication with an intraluminal ultrasound imaging catheter, wherein the processor circuit is configured to:
receive a first plurality of intraluminal ultrasound images obtained by the intraluminal ultrasound imaging catheter during movement of the intraluminal ultrasound imaging catheter within a body lumen of a patient, the body lumen comprising a plurality of segments and a compression within at least one segment;
for at least one segment, based on the first plurality of intraluminal ultrasound images, automatically determine a target location within the compression and a reference location comprising a healthy portion of the lumen; and
output, to a display in communication with the processor circuit, a screen display comprising the target location, the reference location, and at least one quantity associated with the target location and reference location.

2. The system of claim 1, wherein the processor circuit is further configured to:
receive a second plurality of intraluminal ultrasound images obtained by the intraluminal ultrasound imaging catheter during movement of the intraluminal ultrasound imaging catheter within the body lumen, the body lumen comprising a stent aligned longitudinally with the target location;
based on the second plurality of intraluminal ultrasound images, automatically determine a proximal end of the stent, a distal end of the stent, and a location within the stent where the stent is most constricted; and
output, to the screen display, the proximal end of the stent, the distal end of the stent, the location where the stent is most constricted, and at least one quantity associated with the constriction.

3. The system of claim 2, wherein automatically determining at least one of the target location, the reference location, the proximal end of the stent, the distal end of the stent, or the location within the stent where the stent is most constricted involves a machine learning algorithm.

4. The system of claim 1, wherein the body lumen comprises peripheral vasculature and wherein the plurality of segments comprises at least one of a common iliac vein (CIV), an external iliac vein (EIV), a common femoral vein (CFV), or a femoral vein for popliteal access.

5. The system of claim 1, wherein the at least one segment comprises multiple segments.

6. The system of claim 5, wherein the screen display simultaneously shows the target frame and reference frame for each of the multiple segments.

7. The system of claim 1, wherein the target location is determined at least in part by variation of a first lumen metric along the segment, and the reference location is determined at least in part by variation of a second lumen metric along the segment.

8. The system of claim 7, wherein the first lumen metric and the second lumen metric are the same.

9. The system of claim 7, wherein the first lumen metric or the second lumen metric comprises an area, minimum diameter, max diameter, effective diameter, average diameter, aspect ratio, or flow resistance of the lumen.

10. The system of claim 1, wherein the target location or the reference location is determined based at least in part on the intraluminal ultrasound imaging catheter.

11. The system of claim 1, wherein the target location or the reference location is determined based at least in part on a length of the movement of the intraluminal ultrasound imaging catheter within the body lumen.

12. The system of claim 1, wherein the target location or the reference location is determined based at least in part on the segment in which the compression is located.

13. The system of claim 1, wherein the target location or the reference location is determined based at least in part on a border of a second body lumen adjacent to the body lumen.

14. The system of claim 1, wherein the screen display comprises at least one of a roadmap image or an image longitudinal display (ILD).

15. An intraluminal ultrasound imaging method, comprising:
With a processor circuit in communication with an intraluminal ultrasound imaging catheter:
receiving a first plurality of intraluminal ultrasound images obtained by the intraluminal ultrasound imaging catheter during movement of the intraluminal ultrasound imaging catheter within a body lumen of a patient, the body lumen comprising a plurality of segments and a compression within at least one segment;
for at least one segment, based on the first plurality of intraluminal ultrasound images, automatically determining a target location within the compression and a reference location comprising a healthy portion of the lumen proximate to the compression; and
outputting, to a display in communication with the processor circuit, a screen display comprising the target location, the reference location, and at least one quantity associated with the target location and reference location.

16. The method of claim 15, further comprising, with the processor circuit:
receiving a second plurality of intraluminal ultrasound images obtained by the intraluminal ultrasound imaging catheter during movement of the intraluminal ultrasound imaging catheter within the body lumen, the body lumen comprising a stent aligned longitudinally with the target location;
based on the second plurality of intraluminal ultrasound images, automatically determining a proximal end of the stent, a distal end of the stent, and a location within the stent where the stent is most constricted; and
outputting, to the screen display, the proximal end of the stent, the distal end of the stent, the location where the stent is most constricted, and at least one quantity associated with the constriction.

17. The method of claim 15, wherein the body lumen comprises peripheral vasculature and wherein the plurality of segments comprises at least one of a common iliac vein (CIV), an external iliac vein (EIV), or a common femoral vein (CFV).

18. The method of claim 15, wherein the at least one segment comprises multiple segments, and wherein the screen display simultaneously shows the target frame and reference frame for each of the multiple segments.

19. The method of claim 15, wherein the target location is determined at least in part by variation of a first lumen metric along the segment, and the reference location is determined at least in part by variation of a second lumen metric along the segment, wherein the first lumen metric or the second lumen metric comprises an area, minimum diameter, max diameter, effective diameter, average diameter, aspect ratio, or flow resistance of the lumen.

20. The method of claim 19, wherein the first lumen metric and the second lumen metric are the same.
